(19) United States
(12) Patent Application Publication
(10) Pub. No.: US 2024/0081742 A1
(43) Pub. Date: Mar. 14, 2024

(54) DUAL-ANCHOR SENSOR IMPLANT DEVICES

(71) Applicant: EDWARDS LIFESCIENCES CORPORATION, Irvine, CA (US)

(72) Inventors: Michael G. Valdez, Riverside, CA (US); Tiana Tran, Westminster, CA (US)

(21) Appl. No.: 18/509,196

(22) Filed: Nov. 14, 2023

Related U.S. Application Data
(63) Continuation of application No. PCT/US2022/027257, filed on May 2, 2022.
(60) Provisional application No. 63/189,055, filed on May 14, 2021.

(51) Int. Cl. A61B 5/00; A61B 90/00

(52) U.S. Cl. CPC A61B 5/6882; A61B 5/0031; A61B 90/08; A61B 2562/16

(57) ABSTRACT

A sensor implant device includes a sensor device, an anchor base structure secured to the sensor device, a first helical tissue anchor secured to at least one of the sensor device or the anchor base structure, the first helical tissue anchor winding in a first direction, and a second helical tissue anchor secured to at least one of the sensor device or the anchor base structure, the second helical tissue anchor winding in a second direction opposite the first direction.

### RELATED APPLICATION

This application is a continuation of International Patent Application No. PCT/US2022/027257, filed May 2, 2022 and entitled DUAL-ANCHOR SENSOR IMPLANT DEVICES, which claims priority to U.S. Provisional Patent Application No. 63/189,055, filed May 14, 2021 and entitled DUAL-ANCHOR SENSOR IMPLANT DEVICES, the complete disclosure of which is hereby incorporated by reference in its entirety.

### BACKGROUND

### Field

The present disclosure generally relates to the field of medical implant devices.

### Description of Related Art

Various medical procedures involve the implantation of a medical implant devices within the anatomy of the heart. Certain physiological parameters associated with such anatomy, such as fluid pressure, can have an impact on patient health prospects.

### SUMMARY

Described herein are one or more methods and/or devices to facilitate monitoring of physiological parameter(s) associated with certain chambers and/or vessels of the heart, such as the left atrium, or other anatomy or environment, using one or more sensor implant/anchor devices.

In some implementations, the present disclosure relates to a sensor implant device comprising a sensor device, an anchor base structure secured to the sensor device, a first helical tissue anchor secured to at least one of the sensor device or the anchor base structure, the first helical tissue anchor winding in a first direction, and a second helical tissue anchor secured to at least one of the sensor device or the anchor base structure, the second helical tissue anchor winding in a second direction opposite the first direction.

The sensor implant can further comprise a third helical tissue anchor secured to at least one of the sensor device or the anchor base structure, the third helical tissue anchor winding in the first direction. In some embodiments, a tip of the first helical tissue anchor is positioned at a first circumferential position and a tip of the third helical tissue anchor is positioned at a second circumferential position with respect to an axis of the sensor device, the second circumferential position being circumferentially offset from the first circumferential position. For example, the tip of the first helical tissue anchor can be positioned opposite the tip of the third helical tissue anchor with respect to a radius of the sensor implant device.

In some embodiments, the anchor base structure comprises one or more torque-engagement features. For example, the one or more torque-engagement features may comprise one or more radial apertures.

In sonic embodiments, the first helical tissue anchor has a first diameter, the second helical tissue anchor has a second diameter, and the first diameter is greater than the second diameter.

A tissue-engagement portion of the first helical tissue anchor can have a conical helix shape. For example, a tissue-engagement portion of the second helical tissue anchor may have a cylindrical helix shape. In some embodiments, a distal portion of the first helical tissue anchor has a greater pitch than a distal portion of the second helical tissue anchor.

In some embodiments, the anchor base structure comprises a first anchor attachment portion configured to have an attachment portion of the first helical tissue anchor attached thereto and a second anchor attachment portion configured to have an attachment portion of the second helical tissue anchor attached thereto. For example, the first anchor attachment portion can have a diameter that is greater than a diameter of the second anchor attachment portion. In some embodiments, the first anchor attachment portion is associated with a medial portion of the anchor base structure and the second anchor attachment portion is associated with an end portion of the anchor base structure.

In some implementations, the present disclosure relates to a sensor implant device comprising a sensor device comprising a sensor transducer and a wireless transmitter, a first tissue anchor means secured to the sensor device, the first tissue anchor means having a first chirality, and a second tissue anchor means secured to the sensor device, the second tissue anchor means having a second chirality that is opposite the first chirality.

In some embodiments, at least one of the first tissue anchor means or the second tissue anchor means is secured to the sensor device via an anchor base structure. For example, the anchor base structure can be secured to a body of the sensor device. In some embodiments, the anchor base structure comprises a torque-engagement means. For example, the torque-engagement means may comprise at least one of a radial engagement aperture, recess, or edge.

The first chirality can he a left-handed chirality, whereas the second chirality is a right-handed chirality.

In some embodiments, the second issue anchor reaps is disposed, at least in part, radially within the first tissue anchor means.

In some embodiments, the first tissue anchor means and second tissue anchor means are corkscrew anchors. For example, in some embodiments, the first tissue anchor means is conical and the second tissue anchor means is cylindrical.

In some implementations, the present disclosure relates to a sensor implant delivery system comprising a sensor implant device including a housing structure having one or more torque-engagement features, a clockwise helical tissue anchor secured to the housing structure, a counterclockwise helical tissue anchor secured to the housing structure, and a torquing shaft including one or more locking arms configured to engage with at least one of the one or more torque-engagement features of the housing structure.

In some embodiments, the one or more torque-engagement features comprise first and second radial apertures and each of the one or more locking arms of the torquing shaft is configured to radially-inwardly engage with a respective one of the first and second radial apertures.

The torquing shaft may comprise a distal torquing portion and a torque-limiter portion proximally and rotatably coupled to the distal torquing portion, the torque-limiter portion being configured to limit an amount of torque translated from the torque-limiter portion to the distal torquing portion.

In some embodiments, a first one of the distal torquing portion or the torque-limiter portion comprises one or more pegs and a second one of the distal torquing portion or the torque-limiter portion comprises one or more deflectable members configured to engage with the one or more pegs and transfer torque from the one or more pegs to the second one of the distal torquing portion or the torque-limiter portion.

In some embodiments, the distal torquing portion and the torque-limiter portion are coupled by a pin associated with one or more axial retention stoppers.

The system can further comprise an inner sheath configured to be disposed about the torquing shaft and to retain the one or more locking arms in a locking engagement with the one or more torque-engagement features. In some embodiments, the system further comprises an outer sheath configured to have disposed therein the inner sheath, torquing shaft, and sensor implant device.

In some implementations, the present disclosure relates to a method of implanting a sensor implant device. The method comprises advancing a delivery system to a target tissue wall, the delivery system comprising a sensor implant device including a housing structure having one or more torque engagement features, a first helical tissue anchor secured to the housing structure, the first helical tissue anchor winding in a first direction, a second helical tissue anchor secured to the housing structure the second helical tissue anchor winding in a second direction opposite the first direction, and a torquing shaft including one or more locking arms configured to engage with the one or more torque engagement features of the housing structure. The method further comprises rotating the torquing shaft in the first direction to at least partially embed the first helical tissue anchor in the target tissue wall and permitting the sensor implant device to rotate in the second direction to thereby at least partially withdraw the first helical tissue anchor from the target tissue wall and embed the second helical tissue anchor in the target tissue wall.

The method can further comprise retracting a sheath from around a distal portion of the torquing shaft to cause the one or more locking arms to disengage from the one or more torque engagement features of the housing structure.

For purposes of summarizing the disclosure, certain aspects, advantages, and novel features have been described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 illustrates an example representation of a human heart.

FIG. 2 illustrates a superior view of a human heart.

FIG. 3 illustrates example pressure waveforms associated with various chambers and vessels of the heart.

FIG. 4 illustrates a graph showing left atrial pressure ranges.

FIG. 5 is a block diagram representing a sensor implant device in accordance with one or more embodiments.

FIG. 6 is a block diagram representing a system for monitoring one or more physiological parameters associated with a patient according to one or more embodiments.

FIG. 7 illustrates a sensor assembly/device in accordance with one or more embodiments.

FIGS. 8A-8E illustrate perspective, side, top axial, bottom axial, and exploded views, respectively, of a sensor implant device in accordance with one or more embodiments.

FIGS. 9A and 9B show perspective and side views, respectively, of a sensor implant device implanted in tissue in accordance with one or more embodiments.

FIG. 10 shows a sensor implant device including conical helix tissue anchors in accordance with one or more embodiments.

FIG. 11 show a sensor implant device including cylindrical helix tissue anchors in accordance with one or more embodiments.

FIG. 12 shows an exploded view of a sensor implant device in accordance with one or more embodiments.

FIGS. 13A and 13B shows a sensor implant device including an anchor base/housing having a plurality of anchor attachment, portions in accordance with one or more embodiments.

FIG. 14 shows a heart having sensor implant devices implanted in various implantation locations in accordance with one or more embodiments.

FIG. 15 is a cutaway view of a human heart and associated vasculature showing certain catheter access paths for sensor implant device implantation procedures in accordance with one or more embodiments.

FIG. 16 shows a cutaway view of a delivery system for a sensor implant device in accordance with one or more embodiments.

FIGS. 17A and 17B show side and exploded views, respectively, of a torquing shaft in accordance with one or more embodiments.

FIGS. 18A-18C show side, cross-sectional and axial views, respectively, of a distal torquing portion of a torquing shaft in accordance with one or more embodiments.

FIGS. 19A-19C show side, cross-sectional, and axial views, respectively, of a torque-limiting portion of a torquing shaft in accordance with one or more embodiments.

FIG. 20A shows a side view of a coupling between a torquing portion and a torque-limiting portion of a torquing shaft in accordance with one or more embodiments.

FIGS. 20B-20D show axial views of a coupling between a torquing portion and a torque-limiting portion of a torquing shaft in various states in accordance with one or more embodiments.

FIGS. 21-1, 21-2, 21-3, 21-4, and 21-5 provide a flow diagram illustrating a process for implanting a sensor implant device in accordance with one or more embodiments.

FIGS. 22-1 22-2, 22-3, 22-4, and 22-5 provide images of cardiac anatomy and certain devices/systems corresponding to operations of the process of FIGS. 21-1, 21-2, 21-3, 21-4, and 21-5 in accordance with one or more embodiments.

FIGS. 23-1 and 23-2 illustrate various implantation stages/states for a sensor implant device in accordance with one or more aspects of the present disclosure.

FIGS. 24-1 and 24-2 illustrate various implantation stages/states for a sensor implant device in accordance with one or more aspects of the present disclosure.

### DETAILED DESCRIPTION

The headings provided herein are for convenience only an necessarily affect the scope or meaning of the claimed invention.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the embodiments disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Certain standard anatomical terms of location are used herein to refer to certain device components/features and to the anatomy of animals, and namely humans, with respect to the preferred embodiments. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between clement(s)/structures(s), as illustrated in the drawings. it should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or clement; structure, and vice-versa.

The present disclosure relates to systems, devices, and methods for monitoring of one or more physiological parameters of a patient (e.g., blood pressure) using sensor-integrated implant devices configured to anchor into biological tissue. In some implementations, the present disclosure relates to helical tissue anchors and anchor housings that incorporate or are associated with pressure sensors or other sensor devices. The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly. Certain embodiments are disclosed herein in the context of cardiac implant devices. However, although certain principles disclosed herein are particularly applicable to the anatomy of the heart, it should be understood that sensor implant devices in accordance with the present disclosure may be implanted in, or configured for implantation in, any suitable or desirable anatomy.

Sensor implant devices of the present disclosure include opposing anchors, such as anchors having opposite chirality. The use of opposing tissue anchors in connection with sensor implant. devices as disclosed. herein can provide improved tissue engagement and anti-rotation characteristics.

### Cardiac Physiology

The anatomy of the heart is described below to assist the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

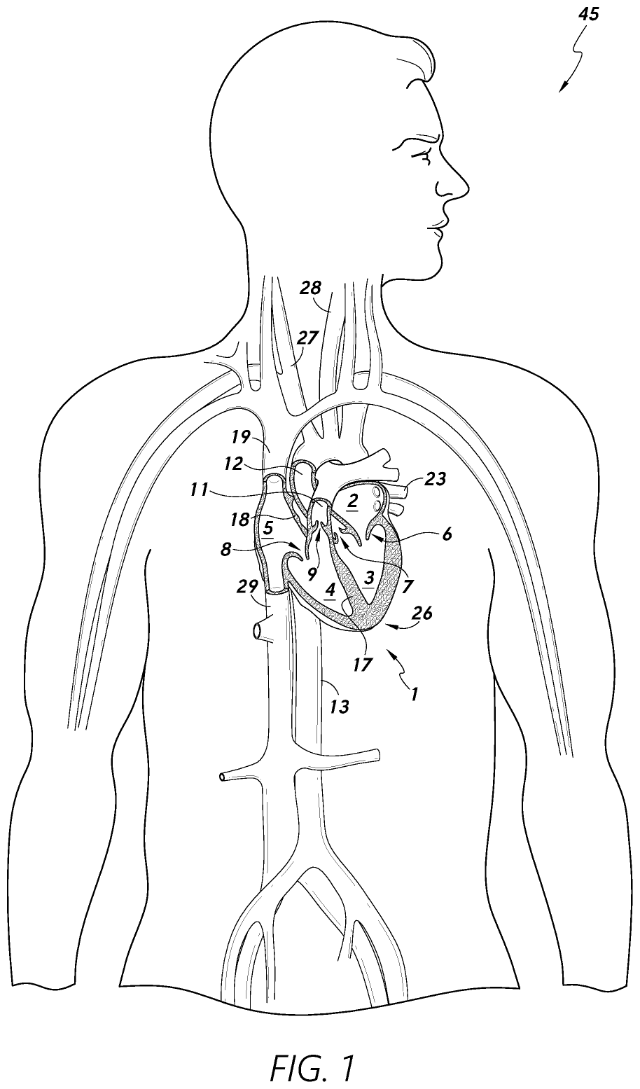

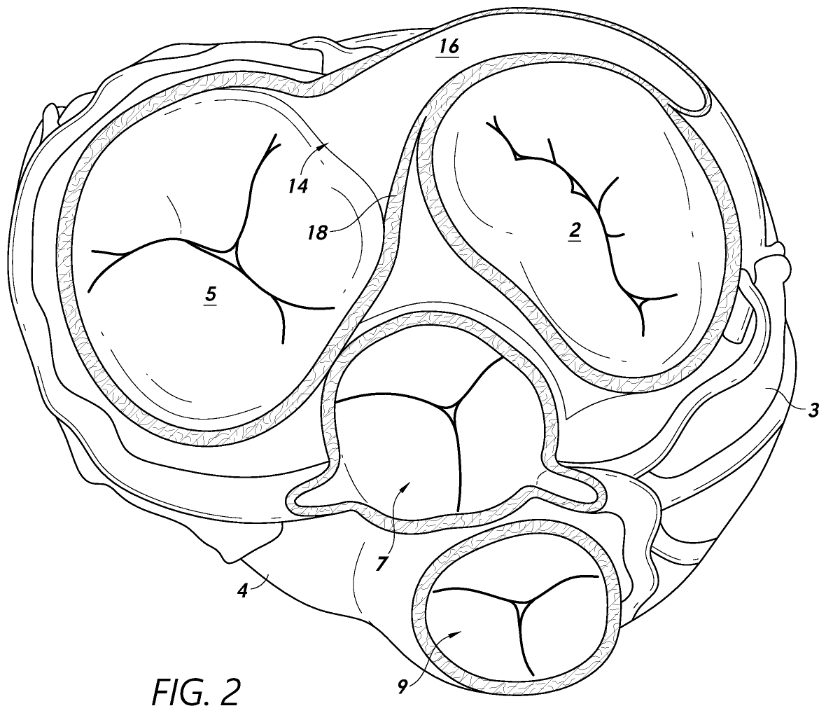

FIGS. 1 and 2 illustrate vertical/frontal and horizontal/superior cross-sectional views, respectively, of an example heart 1 having various features/anatomy relevant to certain aspects of the present inventive disclosure. The heart 1 includes four chambers, namely the left, atrium 2, the left ventricle 3, the right ventricle 4, and the right atrium 5. In terms of blood flow, blood generally flows from the right ventricle 4 into the pulmonary artery 11 via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs.

In addition to the pulmonary valve 9, the heart 1 includes three additional valves for aiding the circulation of blood therein, including the tricuspid valve 8, the aortic valve 7, and the mitral valve 6. The tricuspid valve 8 separates the right atrium 5 from the right ventricle 4. The tricuspid valve 8 generally has three cusps or leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets/cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage. Disfunction of a heart valve and/or associated leaflets (e.g., pulmonary valve disfunction) can result in valve leakage and/or other health complications.

The atrioventricular (i.e., mitral and tricuspid) heart valves may further comprise a collection of chordae tendineae and papillary muscles (not shown) for securing the leaflets of the respective valves to promote and/or facilitate proper coaptation of the valve leaflets and prevent prolapse thereof. The papillary muscles, for example, may generally comprise finger-like projections from the ventricle wall. The valve leaflets are connected to the papillary muscles by the chordae tendineae. A wall of muscle, referred to as the septum, separates the left-side chambers from the right-side chambers. In particular, an atrial septum wall portion 18 (referred to herein as the “atrial septum,” “atrial septum,” or “septum”) separates the left atrium 2 from the right atrium 5, whereas a ventricular septum wall portion 17 (referred to herein as the “ventricular septum,” “interventricular septum.” or “septum”) separates the left ventricle 3 from the right ventricle 4. The inferior tip 14 of the heart 1 is referred to as the apex and is generally located on or near the midclavicular line, in the fifth intercostal space.

### Health Conditions Associated with Cardiac Pressure and Other Parameters

As referenced above, certain physiological conditions or parameters associated with the cardiac anatomy can impact the health of a patient. For example, congestive heart failure is a condition associated with the relatively slow movement of blood through the heart and/or body, which causes the fluid pressure in one or more chambers of the heart to increase. As a result, the heart does not pump sufficient oxygen to meet the body's needs. The various chambers of the heart may respond to pressure increases by stretching to hold more blood to pump through the body or by becoming relatively stiff and/or thickened. The walls of the heart can eventually weaken and become unable to pump as efficiently. In some cases, the kidneys may respond to cardiac inefficiency by causing the body to retain fluid. Fluid build-up in arms, legs, ankles, feet, lungs, and/or other organs can cause the body to become congested, which is referred to as congestive heart failure. Acute decompensated congestive heart failure is a leading cause of morbidity and mortality, and therefore treatment and/or prevention of congestive heart failure is a significant concern in medical care.

The treatment and/or prevention of heart failure (e.g., congestive heart failure) can advantageously involve the monitoring of pressure in one or more chambers or regions of the heart or other anatomy. As described above, pressure buildup in one or more chambers or areas of the heart can be associated with congestive heart failure. Without direct or indirect monitoring of cardiac pressure, it can be difficult to infer, determine, or predict the presence or occurrence of congestive heart failure. For example, treatments or approaches not involving direct or indirect pressure monitoring may involve measuring or observing other present physiological conditions of the patient, such as measuring body weight, thoracic impedance, right heart catheterization, or the like. In some solutions, pulmonary capillary wedge pressure can be measured as a surrogate of left atrial pressure. For example, a pressure sensor may be disposed or implanted in the pulmonary artery, and readings associated therewith may be used as a surrogate for left atrial pressure. However, with respect to catheter-based pressure measurement in the pulmonary artery or certain other chambers or regions of the heart, use of invasive catheters may be required to maintain such pressure sensors, which may be uncomfortable or difficult to implement. Furthermore, certain lung-related conditions may affect pressure readings in the pulmonary artery, such that the correlation between pulmonary artery pressure and left atrial pressure may be undesirably attenuated. As an alternative to pulmonary artery pressure measurement, pressure measurements in the right ventricle outflow tract may relate to left atrial pressure as well. However, the correlation between such pressure readings and left atrial pressure may not be sufficiently strong to he utilized in congestive heart failure diagnostics, prevention, and/or treatment.

Additional solutions may be implemented for deriving or inferring left atrial pressure. For example, the E/A ratio, which is a marker of the function of the left ventricle of the heart representing the ratio of peak velocity blood flow from gravity in early diastole (the E wave) to peak velocity flow in late diastole caused by atrial contraction (the A wave), can be used as a surrogate for measuring left atrial pressure. The E/A ratio may be determined using echocardiography or other imaging technology; generally, abnormalities in the E/A ratio may suggest that the left ventricle cannot fill with blood properly in the period between contractions, which may lead to symptoms of heart failure, as explained above. However, E/A ratio determination generally does not provide absolute pressure measurement values.

Various methods for identifying and/or treating congestive heart failure involve the observation of worsening congestive heart failure symptoms and/or changes in body weight. However, such signs may appear relatively late and/or he relatively unreliable. For example, daily bodyweight measurements may vary significantly (e.g., up to 9% or more) and may be unreliable in signaling heart-related complications. Furthermore, treatments guided by monitoring signs, symptoms, weight, and/or other biomarkers have not been shown to substantially improve clinical outcomes. In addition, for patients that have been discharged, such treatments may necessitate remote telemedicine systems.

The present disclosure provides systems, devices, and methods for guiding the administration of medication relating to the treatment of congestive heart failure at least in part by directly monitoring pressure in the left atrium, or other chamber or vessel for which pressure measurements are indicative of left atrial pressure and/or pressure levels in one or more other vessels/chambers, such as for congestive heart failure patients in order to reduce hospital readmissions, morbidity, and/or otherwise improve the health prospects of the patient.

### Cardiac Pressure Monitoring

Cardiac parameter (e.g., pressure) monitoring in accordance with embodiments of the present disclosure may provide a proactive intervention mechanism for preventing or treating congestive heart failure and/or other physiological conditions. Generally, increases in ventricular filling pressures associated with diastolic and/or systolic heart failure can occur prior to the occurrence of symptoms that lead to hospitalization. For example, cardiac pressure indicators may present weeks prior to hospitalization with respect to some patients. Therefore, pressure monitoring systems in accordance with embodiments of the present disclosure may advantageously be implemented to reduce instances of hospitalization by guiding the appropriate or desired titration and/or administration of medications before the onset of heart failure.

Dyspnea represents a cardiac pressure indicator characterized by shortness of breath or the feeling that one cannot breathe sufficiently. Dyspnea may result from elevated atrial pressure, which may cause fluid buildup in the lungs from pressure back-up. Pathological dyspnea can result from congestive heart failure. However, a significant. amount of time may elapse between the time of initial pressure elevation and the onset of dyspnea, and therefore symptoms of dyspnea may not provide sufficiently-early signaling of elevated atrial pressure. By monitoring pressure directly according to embodiments of the present disclosure, normal ventricular filling pressures may advantageously be maintained, thereby preventing or reducing effects of heart failure, such as dyspnea.

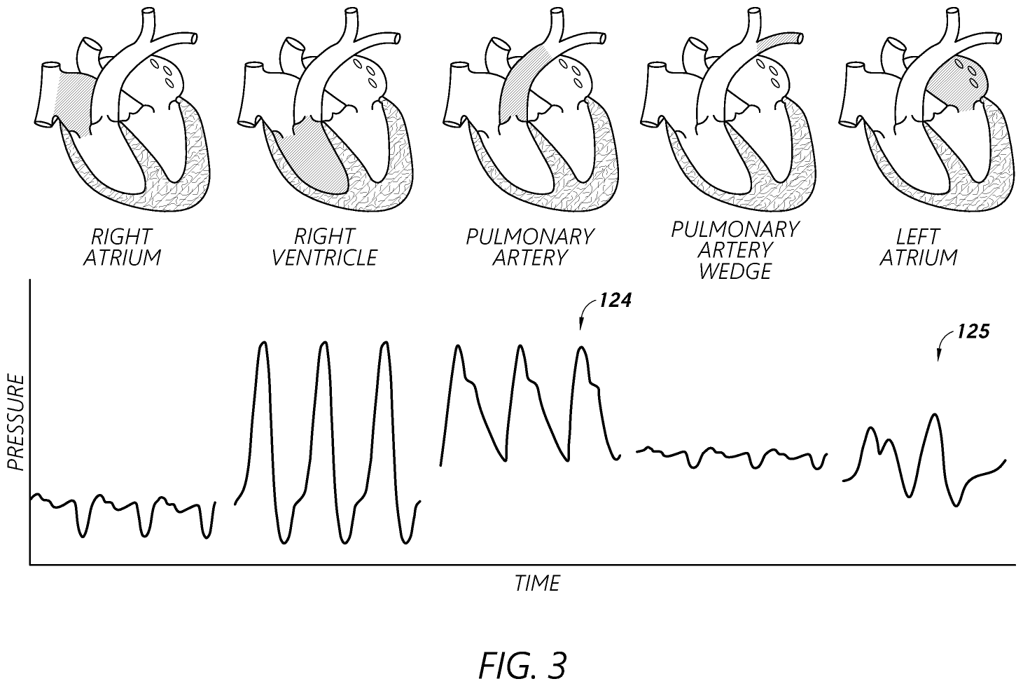

As referenced above, with respect to cardiac pressures, pressure elevation in the left atrium may be particularly correlated with heart failure. FIG. 3 illustrates example pressure waveforms associated with various chambers and vessels of the heart according to one or more embodiments. The various waveforms illustrated. in FIG. 3 may represent waveforms obtained using right heart catheterization to advance one or more pressure sensors to the respective illustrated and labeled chambers or vessels of the heart. As illustrated in FIG. 3, the waveform 125, which represents left atrial pressure, may be considered to provide the best feedback for early detection of congestive heart failure. Furthermore, there may generally be a relatively strong correlation between increases and left atrial pressure and pulmonary congestion.

Left atrial pressure may generally correlate well with left ventricular end-diastolic pressure. However, although left atrial pressure and end-diastolic pulmonary artery pressure can have a significant correlation, such correlation may be weakened when the pulmonary vascular resistance becomes elevated. That is, pulmonary artery pressure generally fails to correlate adequately with left ventricular end-diastolic pressure in the presence of a variety of acute conditions, which may include certain patients with congestive heart failure. For example, pulmonary hypertension, which affects approximately 25% to 83% of patients with heart failure, can affect the reliability of pulmonary artery pressure measurement for estimating left-sided filling pressure. Therefore, pulmonary artery pressure measurement alone, as represented by the waveform 124, may be an insufficient or inaccurate indicator of left ventricular end-diastolic pressure, particularly for patients with co-morbidities, such as lung disease and/or thromboembolism. Left atrial pressure may further be correlated at least partially with the presence and/or degree of mitral regurgitation.

Left atrial pressure readings may be relatively less likely to be distorted or affected by other conditions, such as respiratory conditions or the like, compared to the other pressure waveforms shown in FIG. 3. Generally, left atrial pressure may be significantly predictive of heart failure, such as up two weeks before manifestation of heart failure. For example, increases in left atrial pressure, and both diastolic and systolic heart failure, may occur weeks prior to hospitalization, and therefore knowledge of such increases may be used to predict the onset of congestive heart failure, such as acute debilitating symptoms of congestive heart failure.

Cardiac pressure monitoring, such as left atrial pressure monitoring, can provide a mechanism to guide administration of medication to treat and/or prevent congestive heart failure. Such treatments may advantageously reduce hospital readmissions and morbidity, as well as provide other benefits. An implanted pressure sensor in accordance with embodiments of the present disclosure may be used to predict heart failure up two weeks or more before the manifestation of symptoms or markers of heart failure (e.g., dyspnea). When heart failure predictors are recognized using cardiac pressure sensor embodiments in accordance with the present disclosure, certain prophylactic measures may be implemented, including medication intervention, such as modification to a patient's medication regimen, which may help prevent or reduce the effects of cardiac dysfunction. Direct pressure measurement in the left atrium can advantageously provide an accurate indicator of pressure buildup that may lead to heart failure or other complications. For example, trends of atrial pressure elevation may be analyzed or used to determine or predict the onset of cardiac dysfunction, wherein drug or other therapy may be augmented to cause reduction in pressure and prevent or reduce further complications.

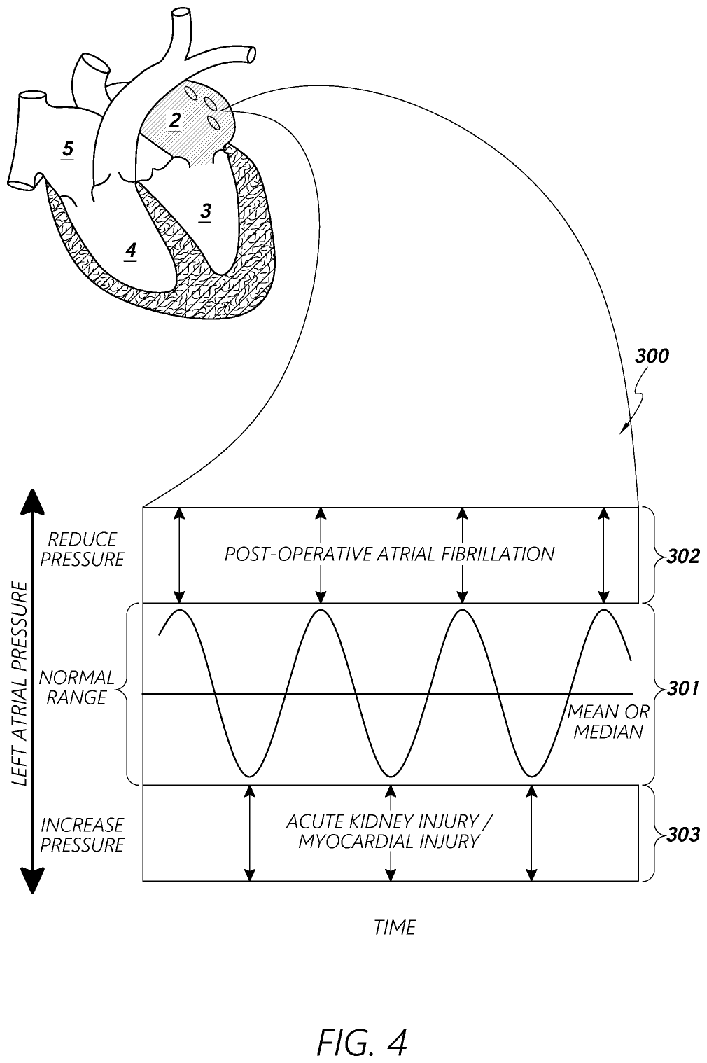

FIG. 4 illustrates a graph 300 showing left atrial pressure ranges including a normal range 301 of left atrial pressure that is not generally associated with substantial risk of postoperative atrial fibrillation, acute kidney injury, myocardial injury, heart failure and/or other health conditions. Embodiments of the present disclosure provide systems, devices, and methods for determining whether a patient's left atrial pressure is within the normal range 301, above the normal range 303, or below the normal range 302 through the use of certain sensor implant devices. For detected left atrial pressure above the normal range, which may be correlated with an increased risk of heart failure, embodiments of the present disclosure as described in detail below can inform efforts to reduce the left atrial pressure until it is brought within the normal range 301. Furthermore, for detected left atrial pressure that is below the normal range 301, which may be correlated with increased risks of acute kidney injury, myocardial injury, and/or other health complications, embodiments of the present disclosure as described in detail below can serve to facilitate efforts to increase the left atrial pressure to bring the pressure level within the normal range 301.

### Sensor Implant Devices

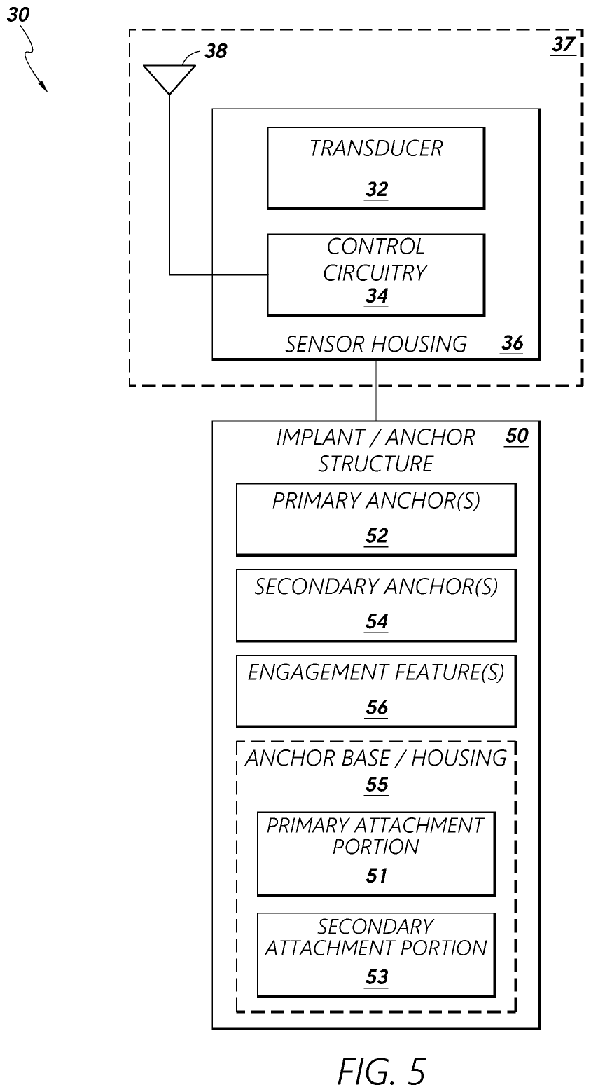

In some implementations, the present disclosure relates to sensors associated or integrated with cardiac implant devices. Such integrated devices may be used to provide controlled and/or more effective therapies for treating and preventing heart failure and/or other health complications related to cardiac function. FIG. 5 is a block diagram illustrating an implant device 30 comprising a helical tissue anchor (or other type of implant) structure 50. In some embodiments, the anchor structure 50 is physically integrated with and/or connected to a sensor device 37. The sensor device 37 may be, for example, a pressure sensor, or other type of sensor. In some embodiments, the sensor 37 comprises a transducer 32. such as a pressure transducer, as well as certain control circuitry 34, which may be embodied in, for example, an application-specific integrated circuit (ASIC).

The control circuitry 34 may be configured to process signals received from the transducer 32 and/or communicate signals associated therewith wirelessly through biological tissue using the antenna 38. The term “control circuitry” is used herein according to its broad and ordinary meaning, and may refer to any collection of processors, processing circuitry, processing modules/units, chips, dies (e.g., semiconductor dies including come or more active and/or passive devices and/or connectivity circuitry), microprocessors, micro-controllers, digital signal processors, microcomputers, central processing units, field-programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further comprise one or more, storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in embodiments in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry. The transducer(s) 32 and/or antenna(s) 38 can be considered part of the control circuitry 34.

The antenna 38 may comprise one or more coils or loops of conductive material, such as copper wire or the like. In some embodiments, at least a portion of the transducer 32, control circuitry 34, and/or the antenna 38 are at least partially disposed or contained within a sensor housing 36, which may comprise any type of material, and may advantageously be at least partially hermetically sealed. For example, the housing 36 may comprise glass or other rigid material in some embodiments, which may provide mechanical stability and/or protection for the components housed therein. In some embodiments, the housing 36 is at least partially flexible. For example, the housing may comprise polymer or other flexible structure/material, which may advantageously allow for folding, bending, or collapsing of the sensor 37 to allow for transportation thereof through a catheter or other introducing means.

The transducer 32 may comprise any type of sensor means or mechanism. For example, the transducer 32 may be a force-collector-type pressure sensor. In some embodiments, the transducer 32 comprises a diaphragm, piston, Bourdon tube, bellows, or other strain- or deflection-measuring component(s) to measure strain or deflection applied over an area/surface thereof. The transducer 32 may be associated with the housing 36, such that at least a portion thereof is contained within or attached to the housing 36. With respect to sensor devices/components being “associated with” a stent or other implant structure, such terminology may refer to a sensor device or component being physically coupled, attached, or connected to, or integrated with, the implant structure.

In some embodiments, the transducer 32 comprises or is a component of a piezoresistive strain gauge, which may be configured to use a bonded or formed strain gauge to detect strain due to applied pressure, wherein resistance increases as pressure deforms the component/material. The transducer 32 may incorporate any type of material, including but not limited to silicon (e.g., monocrystalline), polysilicon thin film, bonded metal foil, thick film, silicon-on-sapphire, sputtered thin film, and/or the like.

In some embodiments, the transducer 32 comprises or is a component of a capacitive pressure sensor including a diaphragm and pressure cavity configured to form a variable capacitor to detect strain due to pressure applied to the diaphragm. The capacitance of the capacitive pressure sensor may generally decrease as pressure deforms the diaphragm. The diaphragm may comprise any material(s), including but not limited to metal, ceramic, silicon, and the like. In some embodiments, the transducer 32 comprises or is a component, of an electromagnetic pressure sensor, which may be configured to measure the displacement of a diaphragm by means of changes in inductance, linear variable displacement transducer (LVDT) functionality, Hall Effect, or eddy current sensing. In some embodiments, the transducer 32 comprises or is a component of a piezoelectric strain sensor. For example, such a sensor may determine strain (e.g., pressure) on a sensing mechanism based on the piezoelectric effect in certain materials, such as quartz.

In some embodiments, the transducer 32 comprises or is a component of a strain gauge. For example, a strain gauge embodiment may comprise a pressure sensitive element on or associated with an exposed surface of the transducer 32. In some embodiments, a metal strain gauge is adhered to a surface of the sensor, or a thin-film gauge may be applied on the sensor by sputtering or other technique. The measuring element or mechanism may comprise a diaphragm or metal foil. The transducer 32 may comprise any other type of sensor or pressure sensor, such as optical, potentiometric, resonant, thermal, ionization. or other types of strain or pressure sensors.

The implant/anchor structure 50 can include a primary anchor 52 and a secondary anchor 54, each of which may include one or more anchor coils/wires (e.g., helical, or ‘corkscrew,’—type tissue anchors), as well as an anchor base/housing, 55 coupled to or otherwise associated with the primary 52 and secondary 54 anchors. While the primary 52 and secondary 54 anchors may each comprise one or more anchor wires, coils, or other elements/members, description herein may refer to primary and secondary anchors in the singular for simplicity. However, it should be understood that any reference herein to a primary or secondary anchor may refer to a single wireform or other anchor element/member, or a plurality of wireforms or other anchor elements/members that collectively constitute the referenced anchor.

The primary anchor 52 may generally have a first type of chirality, which may refer to the handedness/direction of the anchor. The terms “chirality,” “handedness,” and “direction” with respect to a coil and/or anchor are used herein according to their broad and ordinary meanings. For example, with respect to coil-/corkscrew-type wireform tissue anchors, the handedness, or chirality, of the tissue anchor may be considered right-handed, or clockwise, if with respect to a line of sight along the axis of the coil with the proximal end of the tissue anchor facing the observer and the distal end/tip of the coil facing away from the observer, following, the coil in a clockwise direction moves away from the observer and/or towards a distal end/tip of the coil (e.g., pointed tissue-engagement tip with respect to a helical tissue anchor); if movement is towards the observer and/or away from the distal end/tip of the coil (or following the coil in a counterclockwise direction moves away from the observer and/or towards the distal end/tip of the coil), then the chirality/handedness can be considered left-handed, or counterclockwise.

The secondary anchor 54 may generally have a second type of chirality that is different from and/or opposite of the chirality of the primary anchor 52. With opposite/opposing chirality relative to the primary anchor 52, the secondary anchor 54 may have a tendency to refrain from embedding in a relevant tissue wall when the implant/anchor structure 50 is rotated/torqued in the direction of the chirality of the primary anchor 52. That is, when the implant/anchor structure 50 is rotated in a direction to embed the primary anchor 52 in the target tissue, the secondary anchor 54 may tend to remain outside of the tissue and/or back-out/dislodge from the target tissue if already embedded therein to some degree.

When the primary anchor 52 has not been embedded in the target tissue, subsequent backing-out of the primary anchor 52, such as by rotating/rotation of the implant/anchor structure 50 in a direction opposite of the chirality of the primary anchor, such rotation may cause embedding, or further embedding, of the secondary anchor 54 in the target tissue, thereby impeding further unwinding/hacking-out of the primary anchor 52 from the tissue wall and preventing dislodgment of the anchor structure 50 from the tissue wall.

The primary anchor 52 and secondary anchor 54 may each be attached or otherwise secured to the implant/anchor structure 50 and/or the sensor housing 36. For example, one or more of the primary anchor 52 or the secondary anchor 54 may be wrapped around or otherwise engaged with the anchor base/housing 55 of the implant/anchor structure 50. For example, the implant/anchor structure 50 may include an at least partially cylindrical, or other-shaped, housing/base structure to which the primary 52 and/or secondary 54 anchor(s) can be secured, wherein the anchor base/housing 55 is coupled to or otherwise secured to the sensor housing 36 and configured to hold the sensor device 37.

The implant/anchor structure 50 can include certain engagement feature(s) 56 configured to allow for engagement therewith to translate rotational torque from a torquing shaft/device associated with a delivery system used to deliver/implant the device 30 to the implant/anchor structure 50. The engagement feature(s) 56 may comprise one or more apertures against which rotational force may be applied to rotate the implant/anchor structure 50, such as to drive the primary anchor 52 into the target tissue.

In some embodiments, the anchor base/housing 55 includes certain portions configured to have wrapped around, or otherwise attached thereto, portions of the primary 52 and/or secondary 54 anchors. For example, the anchor base/housing 55 may include a primary attachment portion 51, which may comprise a cylindrical surface and/or other feature(s) configured to have wrapped therearound and/or otherwise attached thereto a portion of the primary anchor 52. The anchor base/housing 55 may further comprise a secondary attachment portion 53 configured to have wrapped therearound and/or otherwise attached thereto a portion of the secondary anchor 54. In some embodiments, the primary attachment portion 51 comprises a cylindrical surface and/or other feature(s) that has/have a diameter that is generally larger than that of the secondary attachment portion 53.

Although the implant/anchor structure 50 is shown in FIG. 5 as including the anchor base/housing 55, it should be understood that in some embodiments, such feature(s) may be omitted or different than described above. For example, one or both of the primary 52 and secondary 54 anchors may be attached to the sensor housing 36 rather than a separate anchor base/housing structure. For example, the sensor housing 36 may have associated therewith the engagement feature(s) 56 shown and described. Therefore, it should be understood that references herein to anchor base/housing structures may be understood to refer to features of a sensor housing of the sensor device associated with the relevant embodiment.

By implementing opposing helical coils as the engagement mechanism for tissue anchoring for the sensor implant device 30, dislodgment of the sensor implant device 30 after implantation thereof may be impeded or prevented. For example, the primary anchor 52 may be rotated/wound to penetrate the target tissue, such as the endocardium and/or myocardium of cardiac tissue, until a desired engagement depth is achieved. After penetration of the primary anchor 52, the secondary anchor 54, which may be disposed radially inside one or more portions of the primary anchor, can be pressed and/or glided against the surface of the target tissue (e.g., endocardium). In such a configuration, rotation of the implant/anchor structure 50 in the direction of the chirality of the primary anchor may correspond to rotation of the secondary anchor 54 that is opposite the chirality of the secondary anchor 54, and thus, as the primary anchor 52 is embedded in the tissue during such rotation, the structure 50 is drawn towards the tissue surface. However, as the secondary anchor 52 is not embedded during such rotation, the secondary anchor 52 may become compressed, resulting in a spring force that is generally normal to the surface of the target tissue that pushes sensor implant device 30 and/or structure 50 away from the target tissue surface. When the primary tissue anchor 52 is caused to unwind at least in part, such as due to cardiac motion and/or due to the spring force of the secondary anchor 54, the secondary anchor 54 may engage-in/penetrate the target tissue, thereby creating opposing force/motion that can prevent or impede the further unwinding of the primary anchor 52 from the tissue.

The primary anchor 52, secondary anchor 54, and/or anchor base/housing 55 may comprise any suitable or desirable material, including, but not limited to, memory metal (e.g., Nitinol), stainless steel, polymer, and/or the like. Furthermore, such components may have various configurations and/or sequence of delivery, such as one-piece or two-piece delivery, implantation, and/or configuration. In some implementations, the implant/anchor structure 50 may be delivered and/or implanted prior to placement of the sensor device 37. For example, the sensor device 37 may subsequently be transported to the implantation site after implantation of the implant/anchor structure 50 and coupled to the anchor base/housing 55 to form the sensor implant device 30.

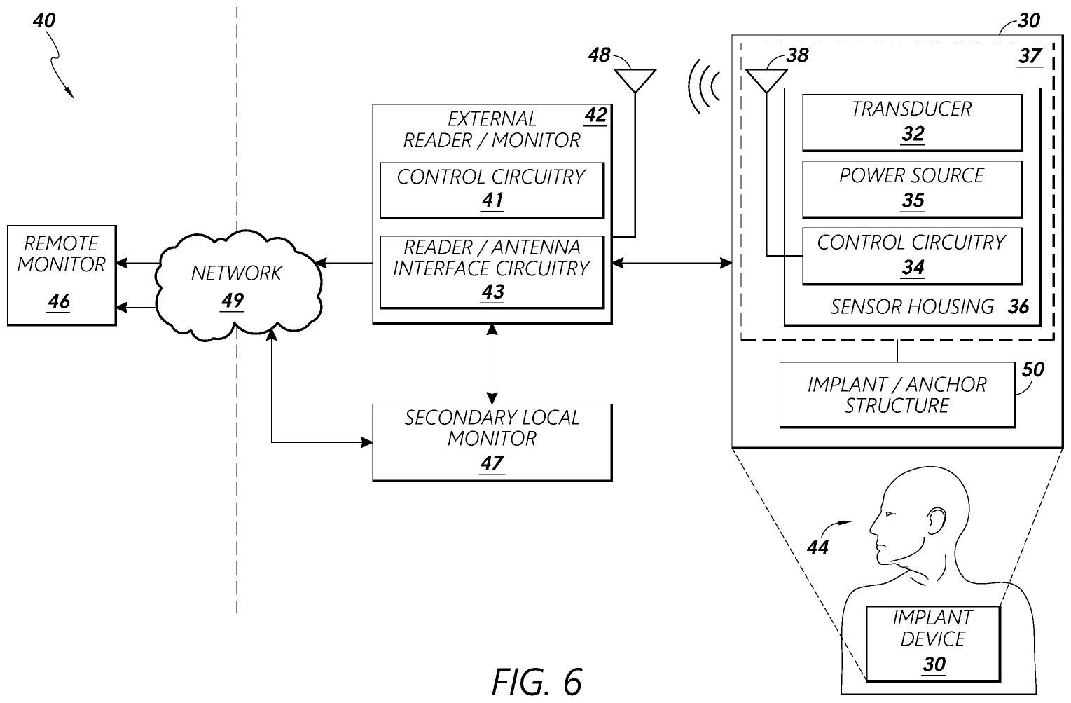

FIG. 6 shows a system 40 for monitoring one or more physiological parameters (e.g., left atrial pressure and/or volume) in a patient 44 according to one or more embodiments. The patient 44 can have a medical implant device 30 implanted in, for example, the heart (not shown), or associated physiology, of the patient 44. For example, the implant device 30 can be implanted at least partially within the left atrium and/or coronary sinus of the patient's heart. The implant device 30 can include one or more sensor transducers 32, such as one or more microelectromechanical system (MEMS) devices (e.g., MEMS pressure sensors, or other type of sensor transducer).

In certain embodiments, the monitoring system 40 can comprise at least two subsystems, including an implantable internal subsystem or device 30 that includes the sensor transducer(s) 32, as well as control circuitry 34 comprising one or more microcontroller(s), discrete electronic component(s), and one or more power and/or data. transmitter(s) 38 (e.g., antennae coil). The monitoring system 40 can further include an external (e.g., non-implantable) subsystem that includes an external reader 42 (e.g., coil), which may include a wireless transceiver that is electrically and/or communicatively coupled to certain control circuitry 41. In certain embodiments, both the internal 30 and external 42 subsystems include a corresponding coil antenna for wireless communication and/or power delivery through patient tissue disposed therebetween. The sensor implant device 30 can be any type of implant device. For example, in some embodiments, the implant device 30 comprises a pressure sensor integrated with another functional implant structure 50, such as a corkscrew tissue anchor device/structure.

Certain details of the implant device 30 are illustrated in the enlarged block 30 shown. The implant device 30 can comprise an implant/anchor structure 50 as described herein. For example, the implant/anchor structure 50 can include a percutaneously-deliverable helical/corkscrew tissue anchor device configured to be secured to and/or in a tissue wall to provide a secure anchoring therein, as described in detail throughout the present disclosure. The implant/anchor structure 50 can comprise primary and secondary opposing helical/coiled wireforms in some embodiments, as disclosed in detail herein.

Although certain components are illustrated in FIG. 6 as pail of the implant device 30, it should be understood that the sensor implant device 30 may only comprise a subset of the illustrated components/modules and can comprise additional components/modules not illustrated. The implant device may represent an embodiment of the implant device shown in FIG. 5, and vice versa. The implant device 30 can advantageously include one or more sensor transducers 32, which can be configured to provide a response indicative of one or more physiological parameters of the patient 44, such as atrial pressure. Although pressure transducers are described, the sensor transducer(s) 32 can comprise any suitable or desirable types of sensor transducer(s) for providing signals relating to physiological parameters or conditions associated with the implant device 30 and/or patient 44.

The sensor transducer(s) 32 can comprise one or more MEMS sensors, optical sensors, piezoelectric sensors, electromagnetic sensors, strain sensors/gauges, accelerometers, gyroscopes, diaphragm-based sensors, and/or other types of sensors, which can be positioned in the patient 44 to sense one or more parameters relevant to the health of the patient. The transducer 32 may be a force-collector-type pressure sensor. In some embodiments, the transducer 32 comprises a diaphragm, piston, bourdon tube, bellows, or other strain- or deflection-measuring component(s) to measure strain or deflection applied over an area/surface thereof. The transducer 32 may be associated with the sensor housing 36, such that at least a portion thereof is contained within, or attached to, the housing 36.

In some embodiments, the transducer 32 comprises or is a component of a strain gauge, which may be configured to use a bonded or formed strain gauge to detect strain due to applied pressure. For example, the transducer 32 may comprise or be a component of a piezoresistive strain gauge, wherein resistance increases as pressure deforms the component/material of the strain gauge. The transducer 32 may incorporate any type of material, including but not limited to silicone, polymer, silicon (e.g., monocrystalline), polysilicon thin film, bonded metal foil, thick film, silicon-on-sapphire, sputtered thin film, and/or the like. In some embodiments, a metal strain gauge is adhered to the sensor surface, or a thin-film gauge may be applied on the sensor by sputtering or other technique. The measuring element or mechanism may comprise a diaphragm or metal foil. The transducer 32 may comprise any other type of sensor or pressure sensor, such as optical, potentiometric, resonant, thermal, ionization, or other types of strain or pressure sensors.

In some embodiments, the transducer 32 comprises or is a component of a capacitive pressure sensor including a diaphragm and pressure cavity configured to form a variable capacitor to detect strain due to pressure applied to the diaphragm. The capacitance of the capacitive pressure sensor may generally decrease as pressure deforms the diaphragm. The diaphragm may comprise any material(s), including but not limited to metal, ceramic, silicone, silicon or other semiconductor, and the like. In some embodiments, the transducer 32 comprises or is a component of an electromagnetic pressure sensor, which may be configured to measures the displacement of a diaphragm by means of changes in inductance, linear variable displacement transducer (LVDT) functionality, Hall Effect, or eddy current sensing. In some embodiments, the transducer 32 comprises or is a component of a piezoelectric strain sensor. For example, such a sensor may determine strain (e.g., pressure) on a sensing mechanism based on the piezoelectric effect in certain materials, such as quartz.

In some embodiments, the transducer(s) 32 is/are electrically and/or communicatively coupled to the control circuitry 34, which may comprise one or more application-specific integrated circuit (ASIC) microcontrollers or chips. The control circuitry 34 can further include one or more discrete electronic components, such as tuning capacitors, resistors, diodes, inductors, or the like.

In certain embodiments, the sensor transducer(s) 32 can be configured to generate electrical signals that can be wirelessly transmitted to a device outside the patient's body, such as the illustrated local external monitor system 42. In order to perform such wireless data transmission, the implant device 30 can include radio frequency (RF) (or other frequency band) transmission circuitry, such as signal processing circuitry and an antenna 38. The antenna 38 can comprise an antenna coil implanted within the patient. The control circuitry 34 may comprise any type of transceiver circuitry configured to transmit an electromagnetic signal, wherein the signal can be radiated by the antenna 38, which may comprise one or more conductive wires, coils, plates, or the like. The control circuitry 34 of the implant device 30 can comprise, for example, one or more chips or dies configured to perform some amount of processing on signals generated and/or transmitted using the device 30. However, due to size, cost, and/or other constraints, the implant device 30 may not include independent processing capability in some embodiments.

The wireless signals generated by the implant device 30 can be received by the local external monitor device or subsystem 42, which can include a reader/antenna-interface circuitry module 43 configured to receive the wireless signal transmissions from the implant device 30, which is disposed at least partially within the patient 44. For example, the module 43 may include transceiver device(s)/circuitry.

The external local monitor 42 can receive the wireless signal transmissions from the implant device 30 and/or provide wireless power to the implant device 30 using an external antenna 48, such as a wand device. The reader/antenna-interface circuitry 43 can include radio-frequency (RF) (or other frequency band) front-end circuitry configured to receive and amplify the signals from the implant device 30, wherein such circuitry can include one or more filters (e.g., band-pass filters), amplifiers (low-noise amplifiers), analog-to-digital converters (ADC) and/or digital control interface circuitry, phase-locked loop (PLL) circuitry, signal mixers, or the like. The reader/antenna-interface circuitry 43 can further be configured to transmit signals over a network 49 to a remote monitor subsystem or device 46, The RF circuitry of the reader/antenna-interface circuitry 43 can further include one or more of digital-to-analog converter (DAC) circuitry, power amplifiers, low-pass filters, antenna switch modules, antennas or the like for treatment/processing of transmitted signals over the network 49 and/or for receiving signals from the implant device 30. In certain embodiments, the local monitor 42 includes control circuitry 41 for performing processing of the signals received from the implant device 30. The local monitor 42 can be configured to communicate with the network 49 according to a known network protocol, such as Ethernet, Wi-Fi, or the like. In certain embodiments, the local monitor 42 comprises a smartphone, laptop computer, or other mobile computing device, or any other type of computing device.

In certain embodiments, the implant device 30 includes some amount of volatile and/or non-volatile data storage. For example, such data storage can comprise solid-state memory utilizing an array of floating-gate transistors, or the like. The control circuitry 34 may utilize data storage for storing sensed data collected over a period of time, wherein the stored data can be transmitted periodically to the local monitor 42 or another external subsystem. In certain embodiments, the implant device 30 does not include any data storage. The control circuitry 34 may be configured to facilitate wireless transmission of data generated by the sensor transducer(s) 32, or other data associated therewith. The control circuitry 34 may further be configured to receive input from one or more external subsystems, such as from the local monitor 42, or from a remote monitor 46 over, for example, the network 49. For example, the implant device 30 may be configured to receive signals that at least partially control the operation of the implant device 30, such as by activating/deactivating one or more components or sensors, or otherwise affecting operation or performance of the implant device 30.

The one or more components of the implant device 30 can be powered by one or more power sources 35. Due to size, cost and/or electrical complexity concerns, it may be desirable for the power source 35 to be relatively minimalistic in nature. For example, high-power driving voltages and/or currents in the implant device 30 may adversely affect or interfere with operation of the heart or other body part associated with the implant device. In certain embodiments, the power source 35 is at least partially passive in nature, such that power can be received from an external source wirelessly by passive circuitry of the implant device 30, such as through the use of short-range, or near-field wireless power transmission, or other electromagnetic coupling mechanism. For example, the local monitor 42 may serve as an initiator that actively generates an RF field that can provide power to the implant device 30, thereby allowing the power circuitry of the implant device to take a relatively simple form factor. In certain embodiments, the power source 35 can be configured to harvest energy from environmental sources, such as fluid flow, motion, or the like. Additionally or alternatively, the power source 35 can comprise a battery, which can advantageously be configured to provide enough power as needed over the monitoring period (e.g., 3, 5, 10, 20, 30, 40, or 90 days, or other period of time).

In some embodiments, the local monitor device 42 can serve as an intermediate communication device between the implant device 30 and the remote monitor 46. The local monitor device 42 can be a dedicated external unit designed to communicate with the implant device 30. For example, the local monitor device 42 can be a wearable communication device, or other device that can be readily disposed in proximity to the patient 44 and implant device 30. The local monitor device 42 can be configured to continuously, periodically, or sporadically interrogate the implant device 30 in order to extract or request sensor-based information therefrom. In certain embodiments, the local monitor 42 comprises a user interface, wherein a user can utilize the interface to view sensor data, request sensor data, or otherwise interact with the local monitor system 42 and/or implant device 30.

The system 40 can include a secondary local monitor 47, which can be, for example, a desktop computer or other computing device configured to provide a monitoring station or interface for viewing and/or interacting with the monitored cardiac pressure data. In an embodiment, the local monitor 42 can be a wearable device or other device or system configured to be disposed in close physical proximity to the patient and/or implant device 30, wherein the local monitor 42 is primarily designed to receive/transmit signals to and/or from the implant device 30 and provide such signals to the secondary local monitor 47 for viewing, processing, and/or manipulation thereof. The external local monitor system 42 can be configured to receive and/or process certain metadata from or associated with the implant device 30, such as device ID or the like, which can also be provided over the data coupling from the implant device 30.

The remote monitor subsystem 46 can be any type of computing device or collection of computing devices configured to receive, process and/or present monitor data received over the network 49 from the local monitor device 42, secondary local monitor 47, and/or implant device 30. For example, the remote monitor subsystem 46 can advantageously be operated and/or controlled by a healthcare entity, such as a hospital, doctor, or other care entity associated with the patient 44. Although certain embodiments disclosed herein describe communication with the remote monitor subsystem 46 from the implant device indirectly through the local monitor device 42, in certain embodiments, the implant device 30 can comprise a transmitter capable of communicating over the network 49 with the remote monitor subsystem 46 without the necessity of relaying information through the local monitor device 42.

In some embodiments, at least a portion of the transducer 32, control circuitry 34, power source 35 and/or the antenna 38 are at least partially disposed or contained within the sensor housing 36, which may comprise any type of material, and may advantageously be at least partially hermetically sealed. For example, the housing 36 may comprise glass or other rigid material in some embodiments, which may provide mechanical stability and/or protection for the components housed therein. In some embodiments, the housing 36 is at least partially flexible. For example, the housing may comprise polymer or other flexible structure/material, which may advantageously allow for folding, bending, or collapsing of the sensor 30 to allow for transportation thereof through a catheter or other percutaneous introducing means.

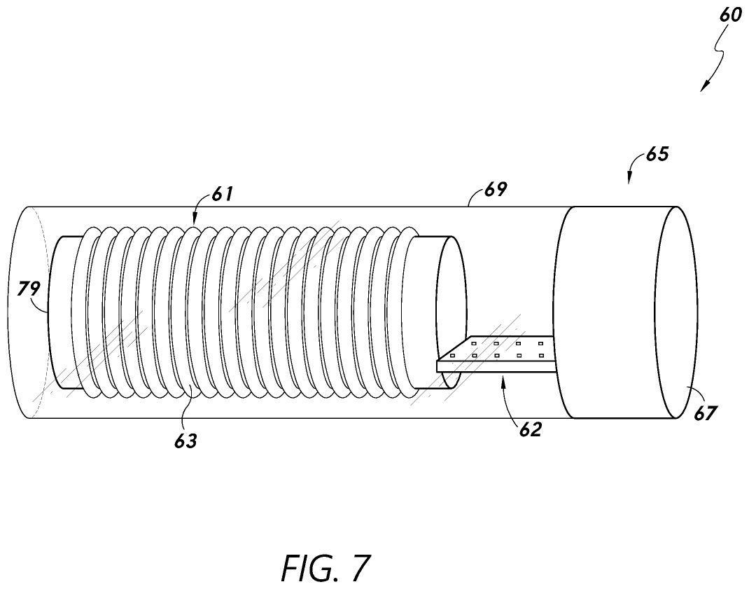

As referenced above, implant devices/structures may be integrated with sensor, antenna/transceiver, and/or other components to facilitate in vivo monitoring of pressure and/or other physiological parameter(s). Sensor devices in accordance with embodiments of the present disclosure may be integrated with tissue anchor structures/devices using any suitable or desirable attachment or integration mechanism or configuration. FIG. 7 illustrates an example sensor assembly/device 60 that can be a component of a sensor implant device, such as the sensor implant device 70 shown in FIG. 7.

With reference to FIG. 7, which shows a detailed view of an example embodiment of a sensor device 60 that may be associated with any of the sensor implant devices disclosed herein, in some embodiments, the sensor device/assembly 60 includes a sensor transducer component 65 and an antenna component 61. The sensor transducer component 65 may comprise any type of sensor transducer as described in detail above. In some embodiments, the sensor device 60 may be attached to or integrated with an opposing-coil tissue anchor structure as described in detail herein.

The sensor transducer component 65 includes a sensor element 67, such as a pressure sensor transducer/membrane. As described herein, the sensor device 60 may be configured to implement wireless data and/or power transmission. The sensor device 60 may include the antenna component 61 for such purpose. The antenna 61, as well as one or more other components of the sensor device 60, may be contained at least partially within a sensor body housing 69, which may further have disposed therein certain control circuitry 62 configured to facilitate wireless data and/or power communication functionality. In some embodiments, the antenna component 61 comprises one or more conductive coils/winds 67, which may facilitate inductive powering and/or data transmission. In embodiments comprising conductive coil(s), such coil(s) may be wrapped/disposed at least partially around a magnetic (e.g., ferrite, iron) core 79.

The sensor device 60 may advantageously be biocompatible. For example, the body/housing 69 may advantageously be biocompatible, such as a housing comprising glass or other biocompatible material. However, at least a portion of the sensor transducer element/membrane 67, such as a diaphragm or other component, may be exposed to the external environment in some embodiments in order to allow for pressure readings, or other parameter sensing, to be implemented. The body/housing 69 may comprise an at least partially rigid cylindrical or tube-like form, such as a glass cylinder form. In some embodiments, the sensor transducer component 65/67 is approximately 3 mm or less in diameter. The antenna 61 may be approximately 20 mm or less in length.

The sensor device 60 may be configured to communicate with an external system when implanted in a heart or other area of a patient's body. For example, the antenna 61 may receive power wirelessly from the external system and/or communicate sensed data or waveforms to and/or from the external system. The sensor element 67 may comprise a pressure transducer. For example, the pressure transducer may be a microelectromechanical system (MEMS) transducer comprising a semiconductor diaphragm component. In some embodiments, the transducer may include an at least partially flexible or compressible diaphragm component, which may be made from silicone or other flexible material. The diaphragm component may be configured to be flexed or compressed in response to changes in environmental pressure. The control circuitry 62 may be configured to process signals generated in response to said flexing/compression to provide pressure readings. In some embodiments, the diaphragm component is associated with a biocompatible layer on the outside surface thereof, such as silicon nitride (e.g., doped silicon nitride) or the like. The diaphragm component and/or other components of the pressure transducer 67 may advantageously be fused or otherwise sealed to/with the body/housing 69 of the sensor device 60 in order to provide hermetic sealing of at least some of the sensor components.

The control circuitry 62 may comprise one or more electronic application-specific integrated circuit (ASIC) chips or die, which may be programmed and/or customized or configured to perform monitoring functionality as described herein and/or facilitate transmission of sensor signals wirelessly. The antenna 61 may comprise a ferrite core 79 wrapped with conductive material in the form of a plurality of coils/winds 63 (e.g., wire coil). In some embodiments, the coils/winds comprise copper or other metal. The antenna 61 may advantageously be configured with coil geometry that does not result in substantial displacement or heating in the presence of magnetic resonance imaging. In some implementations, the sensor implant device 70 may be delivered to a target implant site using a delivery catheter (not shown), wherein the delivery catheter includes a cavity or channel configured to accommodate the advancement of the sensor device 60 therethrough.

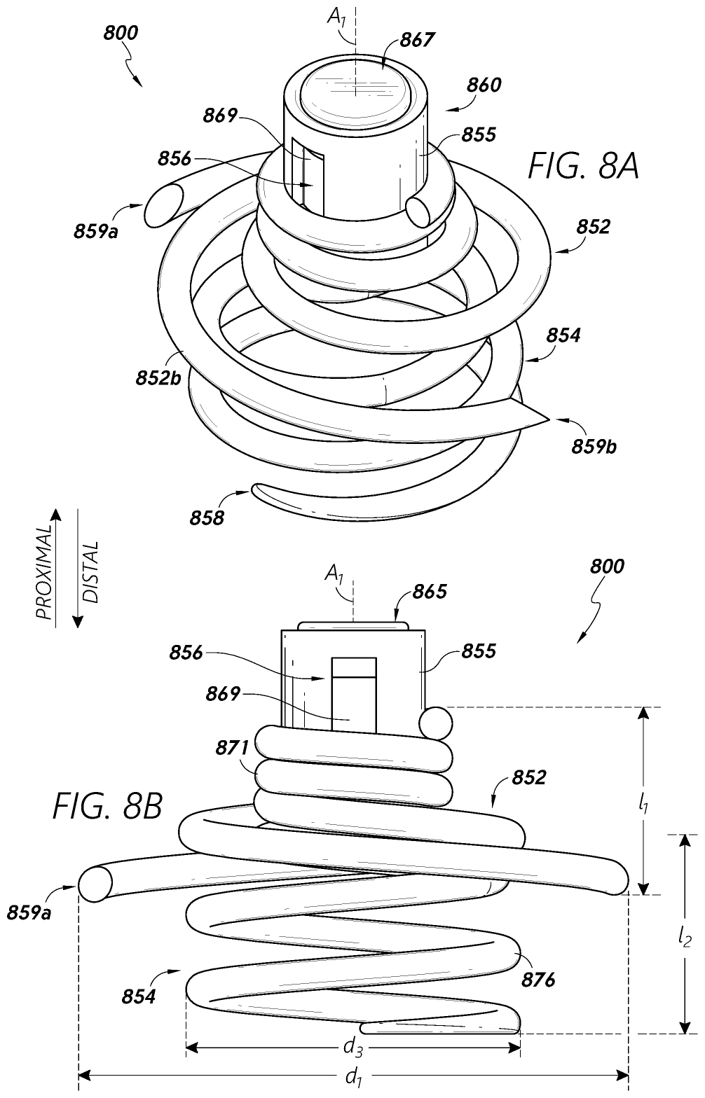

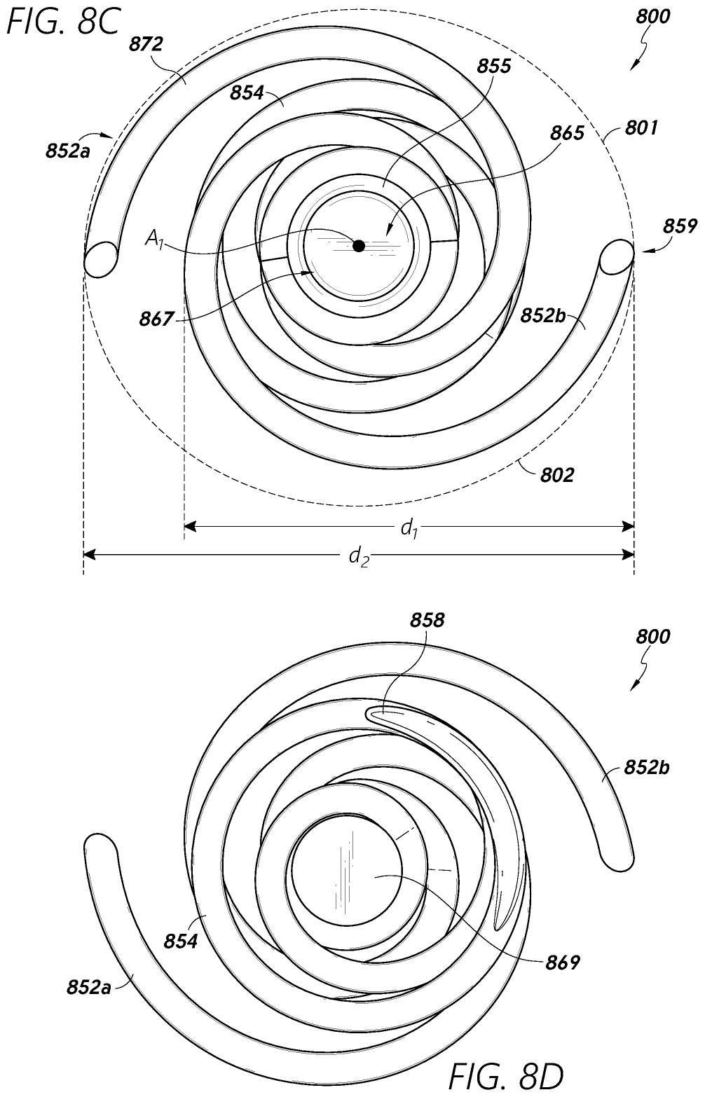

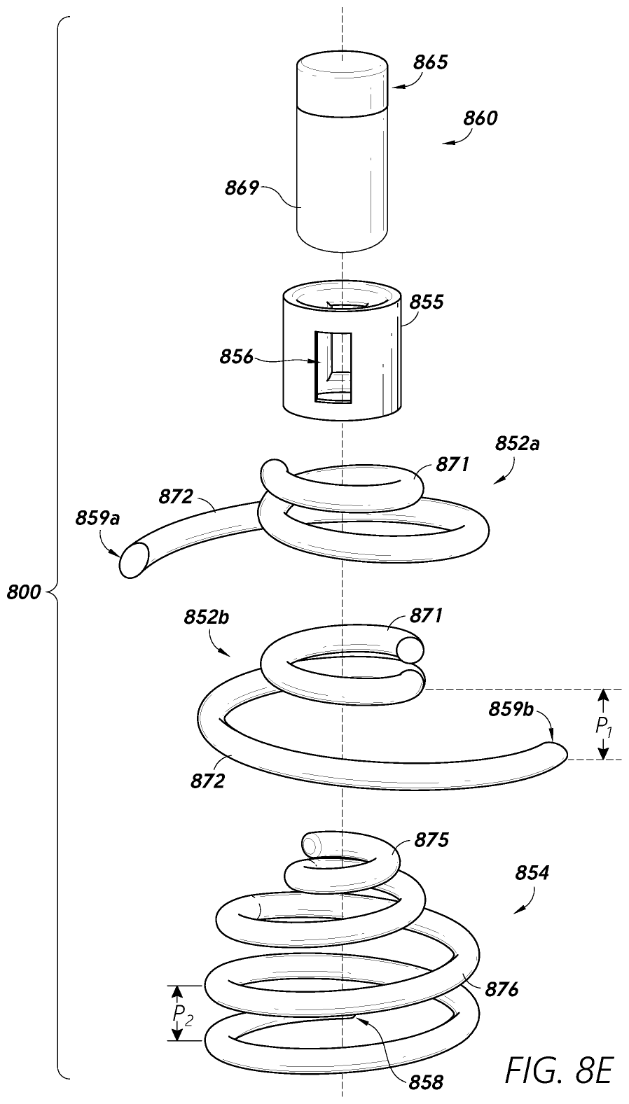

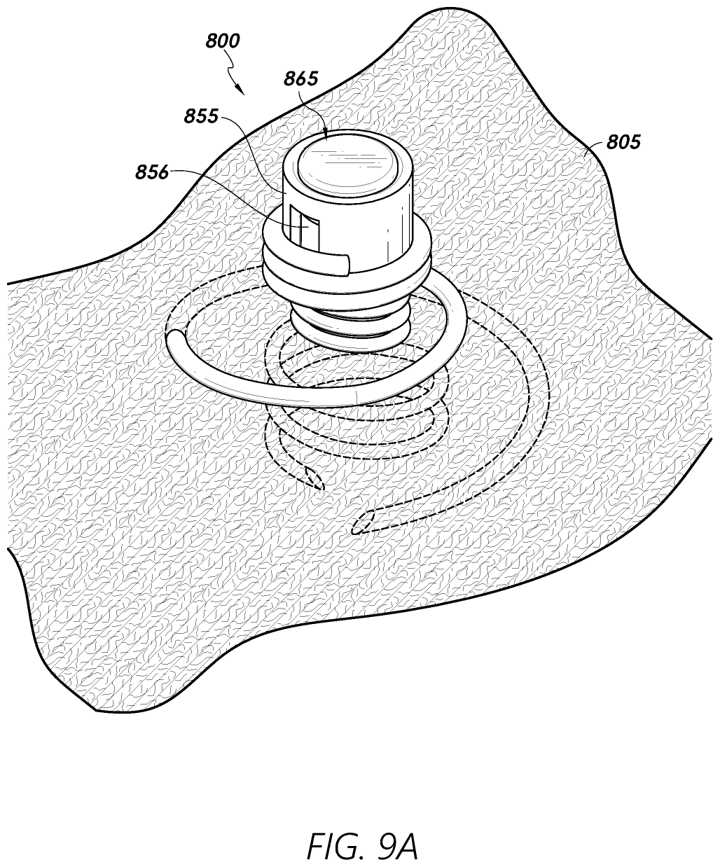

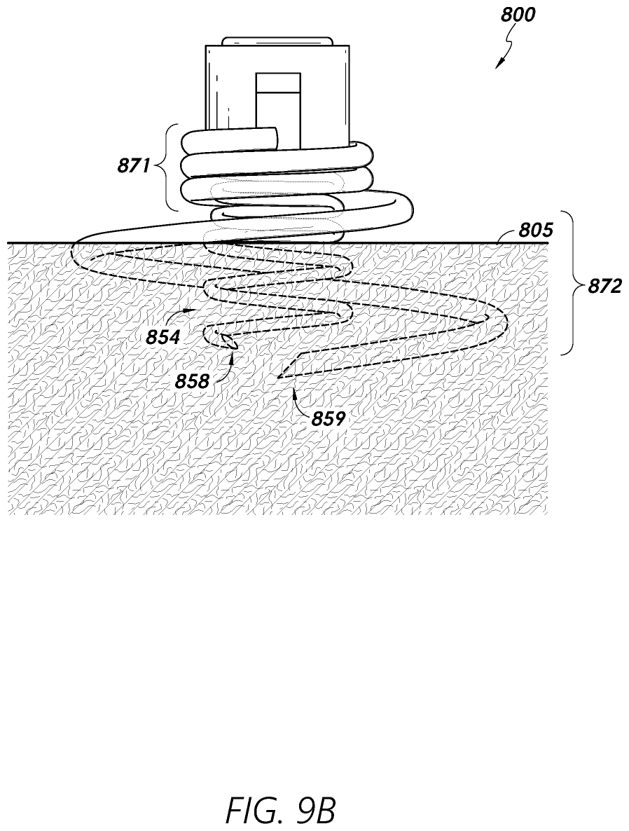

FIGS. 8A-8E illustrate perspective, side, top axial, bottom axial, and exploded views, respectively, of a sensor implant device 800 in accordance with one or more embodiments of the present disclosure. FIGS. 9A and 9B show perspective and side views, respectively, of the sensor implant device 800 for FIGS. 8A-8E implanted in tissue 805 in accordance with one or more embodiments.

The sensor implant device 800 includes a sensor device 860, which may be a cylindrical sensor device as shown in FIG. 7 and described in detail above. The sensor device 800 can include a sensor body/housing, 869 housing certain circuitry of the sensor device 860. Although cylindrical sensor housings and devices are described herein, it should be understood that the sensor device 860 may have any suitable or desirable shape and/or configuration.

The sensor implant device 800 includes an anchor base/housing 855, which may be secured to the sensor device 860 in some manner. For example, in some embodiments, the sensor device 860 may be placed within the anchor base/housing 855, wherein the sensor device 860 is held therein through a friction fit, and/or other attachment means, such as one or more tabs, latches, hooks, edges, flanges, clasps, adhesives, bands, straps, channels, and/or other attachment means or mechanism(s). The sensor 860 may be disposed within the anchor base/housing 855, or the sensor housing 869 may be integrated with the anchor base/housing 855, such that the base/housing 855 is part of the sensor device 860.

The sensor 860 can be configured to self-latch in the anchor base/housing 855 and/or in/to one or more tissue anchor(s) 852, 854 associated therewith, which are described in detail below. For example, the sensor 860 may have one or more ear-type or other protrusions/projection features that are configured to latch into one or more corresponding mating features of the anchor base/housing 855. Although described as a sensor implant device, it should be understood that the implant device 800 may be any type of implantable device, such as an occluder device, therapeutic drug dispenser device, electrical lead, or the like.

The anchor base/housing, 855 may comprise a cylindrical form. In some embodiments, the anchor base/housing 855 includes one or more torque-engagement features 856, which may comprise radial engagement aperture(s) or other window, recess, slot, edge, divot, or similar features configured to allow for application to a surface thereof of rotational force to effect rotation of the anchor base/housing 855 about its axis A₁, thereby rotating the associated components, including the sensor device 860, as well as one or more tissue anchors as described in detail herein. A torquing catheter or shaft may be configured to torque against the window/aperture(s) 856 to drive rotation of the sensor implant device 800.

The sensor implant device 800 includes a primary anchor 852, which may comprise one or more coil wireforms, which may be referred to individually and/or collectively as an anchor or anchors. The primary anchor 852 can have a helical corkscrew-/coil-type form configured to be wound in a direction associated with a chirality thereof to cause the tissue anchor 852 to embed in tissue that is in contact with one or more distal tips (e.g., pointed/sharp tips) 859 thereof. For example, in the illustrated embodiment of FIGS. 8A-8E, the primary anchor 852 has left-handed chirality, such that rotation of the sensor implant device 800 (e.g., by applying rotational torque to the anchor base/housing 855) in a counterclockwise direction can cause the anchor 852 to embed in tissue against which the tip(s) 859 is/are pressed or held. Although left-handed chirality is illustrated for the primary anchor 852, it should be understood that the primary anchor 852 can have any type of chirality, such as right-handedness.

In some embodiments, the primary anchor 852 includes a first coil 852a and a second coil 852b, wherein such coils may have the same/common chirality, such that winding in a given direction associated with the chirality of the coils causes both the coils 852a, 852b to embed in the relevant tissue/material. The two coil portions 852a, 852b may be at least partially intertwined and/or wound together, as shown. Additionally or alternatively, one of the coils 852a, 852b may be configured to wind outside of the other.

One or both of the primary coils 852a, 852b may have respective attachment portions configured to wrap around the base/housing 855 and/or otherwise be attached or secured thereto. Each of the primary coils 852a, 852b may further comprise a tissue-engagement portion 872 configured to be embedded in the relevant target tissue when wound in accordance with the chirality thereof. Tissue-engagement portions of embodiments of the present disclosure may have a helix/helical shape, as illustrated in various figures presented herewith. Although shown as two separate coils 852a, 852b, it should be understood that the primary coil 852 may comprise a single coil in some embodiments. Therefore, any description herein of multiple-/two-coil anchors may be understood to apply to a single-coil embodiment. In two-coil implementations of the primary anchor 852, the separate coils 852a, 852b can be identical or similar, wherein the coils are attached to the base/housing 855 in a rotationally-offset configuration, such as 180° rotated relative to one another.

The primary anchor 852 may have a conical/conic helical form, wherein the coils thereof expand in diameter moving from the proximal to the distal portions thereof, as shown in FIGS. 8A-AE. For example, the tissue engagement portion 872 of the primary coil 852 can spiral radially outward moving along the coil towards the distal tip 859 thereof. The outer perimeter 802 of the primary coil 852, as defined by a radius aligned with the outer- and/or distal-most portions of the primary anchor 852 with respect to an axis A₁ of the implant device 800 and/or sensor device 860, is identified in FIG. 8C, wherein the perimeter 802 may define the diameter d₁ of the sensor implant device 800 and/or primary coil 852. The sensor implant device 800 and/or primary coil 852 may have any suitable or desirable diameter. With respect to multi-coil implementations of the primary 852 and/or secondary 854 tissue anchors, an individual coil component (e.g., 852b) may have a diameter d₂ that is less than the overall diameter d₁ of the sensor implant device 800 and/or associated coil (e.g., primary or secondary). The primary anchor 852 may have any suitable or desirable axial length l₁, a proximal portion 871 of which may constitute an attachment portion of the anchor 852, whereas a distal portion 872 may constitute a tissue-engagement portion 872 of the anchor 852.

The sensor implant device 800 further includes a secondary anchor 854, which may comprise one or more coil wireforms. In the illustrated embodiment, the primary coil 852 comprises multiple coils/components, whereas the secondary anchor 854 comprises only a single coil. However, it should be understood that either or both of the primary 852 and secondary 854 anchors can include one, two, or more coil components. The secondary anchor 854 may have an axial length 12 that, in some embodiments, may be longer (or shorter) compared to the axial length of the primary anchor 852. References herein to axial lengths of tissue anchors may generally be understood to refer to uncompressed configurations of such anchors, as shown in FIGS. 8A-8E. The primary 852 and/or secondary 854 anchors can be welded or bonded in some manner to the housing 855.

The secondary anchor 854 can advantageously have opposing chirality with respect to the primary anchor 852. For example, where the primary anchor 852 has left-handed chirality, as shown in the illustrated embodiment of FIGS. 8A-8E, the secondary anchor 854 may have right-handed chirality, as shown. Alternatively, the primary anchor 852 may have right-handed chirality, whereas the secondary anchor 854 has left-handed chirality. During an implantation process, the primary anchor 852 may be embedded in the target tissue by rotating the sensor implant device 800 in a direction in accordance with the chirality of the primary anchor 852. When the sensor implant device 800 is released from the delivery system, the primary anchor 852 may have a tendency to unwind and/or back-out of the target tissue to some degree, which may cause the secondary anchor 854 to embed into the target tissue.

The secondary anchor 854 may be configured to be attached to the anchor base/housing 855 and/or to the sensor housing 869 of the sensor device 860. For example, the secondary anchor 854 can include an attachment portion 875 configured to wrap around or otherwise secured to a portion of the anchor base/housing 855 and/or housing 869 of the sensor device 860. The tissue-engagement portion 876 of the secondary anchor 854 may be generally cylindrical with respect to the helical shape thereof. For example, in some embodiments, the primary anchor 852 has a spiral/conical helical shape, whereas the secondary anchor 854 has a cylindrical helical shape with respect to respective tissue-engagement portions thereof.

The primary anchor coil can have a pitch p₁, which may generally refer to the height/distance of the coil 852 between complete turns of the helical form thereof in the area of the distal end 859 of the coil 852. In some embodiments, the secondary anchor 854 has a pitch p₂ that is less than the pitch p₁ of the primary coil 852, as best illustrated in FIG. 9B. Alternatively, the pitch p₂ of the secondary coil 854 may be greater than the pitch p₁ of the primary coil 852 in some embodiments.

The secondary coil 854 may have a diameter d₃ that is less than the diameter d₁ of the primary coil 852. Such disparity in diameter between the primary 852 and secondary 854 anchors can allow for simultaneous embedding of the primary 852 and secondary 854 anchors in the relevant tissue, wherein the tissue-engagement portion 872 of the primary anchor 852 can be configured to embed in the target tissue in a manner as to be outside of the tissue-engagement portion 876 of the secondary anchor 854 when the secondary anchor 854 is at least partially embedded in the tissue 805, as shown in FIG. 9B.

The primary 852 and/or secondary 854 anchors can be welded underneath/to the base/housing 855 or can be wrapped around the outside of the base/housing 855 or the sensor housing 869. In some embodiments, the primary anchor 852 is wrapped around a length of the base/housing 855, wherein where the winding of the primary anchor ends, the secondary anchor 854 may be wound around the base/housing 855 distal of the primary anchor. Either or both of the primary 852 and secondary 854 anchors may be wrapped around the anchor base/housing 855 one or more revolutions.

Generally, the attachment portion 871 of the primary anchor 852 may have a tighter coil and/or smaller diameter than that of the tissue-engagement portion 872. Likewise, the attachment portion 875 of the secondary anchor 854 can have a tighter coil and/or smaller diameter than that of the tissue-engagement portion 876. By implementing anchors that have expanded-diameter tissue-engagement portions relative to the respective attachment portions that attached to the anchor base/housing 855 and/or sensor housing 869, the anchors can advantageously provide increased stability for anchoring. For example, the expanded diameters of the respective tissue-engagement portions of the anchors 852, 854 can distribute the anchor load over a greater area/volume of tissue, thereby providing a more stable seating/attachment for the sensor implant device 800 in the tissue wall 805.

A process for implanting the sensor implant device 800 can involve initially torquing the device 800 in the direction associated with the chirality of the primary anchor 852, thereby embedding at least a portion of the tissue-engagement portion 872 of the primary anchor 852 into the target tissue. During such embedding of the primary anchor 852, the secondary anchor 854 may be un-inclined to embed in the tissue, as such rotation may be counter to the chirality of the secondary anchor 854. Therefore, as the primary anchor 852 is embedded in the target tissue, the anchor base/housing 855 and/or sensor device 860 may generally be drawn towards the target tissue, wherein the secondary anchor is thereby compressed at least partially between at least a portion of the anchor base/housing 855 and the target tissue surface. Compression of the secondary anchor 854 can produce a spring force pushing generally away from the target tissue surface, which may cause the primary anchor 852 to unwind to some degree and/or back-out from the target tissue at least in part. Such unwinding may cause the implant device 800 to rotate counter to the chirality of the primary anchor 852, such rotation being associated with the chirality of the secondary anchor 854, and therefore may cause the secondary anchor 854 and distal tip 858 thereof to be embedded at least partially into the target tissue. Such embedding of the secondary anchor can secure the sensor implant device 800 to the target tissue. Therefore, the spring compression of the secondary anchor 854 can serve to further secure the implant device 800 to the target tissue 805 by facilitating partial unwinding of the primary anchor 852 and corresponding winding-in of the secondary anchor 854 Furthermore, the opposing primary and secondary anchor scheme associated with the embodiment(s) shown in FIGS. 8A-9B and described in detail throughout the present disclosure can account for jostling or movement of the sensor implant device after implantation, such that any such movement may be restrained and/or compensated for by causing embedding of the secondary anchor to compensate for backing-out of the primary anchor experienced by the implant device 800 that results in further embedding of either the primary 852 or the secondary 854 anchor.

Although the tissue anchor 854 is described herein as the secondary anchor, whereas the outer tissue anchor 852 is described as the primary anchor, it should be understood that in some configurations, the anchor 854 may be the primary tissue anchor, whereas the outer anchor 852 is the secondary tissue anchor. For example, implantation of the sensor implant device 800 may comprise first rotating the sensor implant device in a direction in accordance with the chirality of the tissue anchor 854, wherein after implantation, the anchor 854 may back-out to some degree, thereby causing rotation of the sensor implant device 800 in a direction opposite of the chirality of the tissue anchor 854 and in accordance with the chirality of the tissue anchor 852, thereby causing the tissue anchor 852 to be embedded at least partially into the target tissue and prevent further backing out of the anchor 854, thereby securing the sensor implant device 800 in place. in some implementations, the primary and secondary anchors may be implanted in connection with separate, subsequent steps of the implantation procedure. For example, the primary anchor may be implanted together with the sensor and/or anchor housing/base, whereas the secondary coil may be implanted after embedding the primary coil in the target tissue.

FIG. 10 shows a sensor implant device 1000 including primary 1052 and secondary 1054 anchors, wherein both the primary 1052 and secondary 1054 anchors have a conical shape with respect to tissue-engagement portions thereof. That is, as with the embodiment shown in FIGS. 8A-9B, the primary anchor 1052 can have an outwardly-spiraling tissue-engagement portion 1072 moving from proximal to distal portions thereof. Furthermore, as an alternative to the illustrated embodiment shown in FIGS. 8A-9B, the secondary anchor 1054 may likewise spiral outwardly moving from proximal to distal portions thereof. The cone form/shape of the secondary anchor 1054 may be dimensioned and configured to fit within the cone form/shape of the primary anchor 1052 to allow for simultaneous presence and/or embedding of the respective anchors.

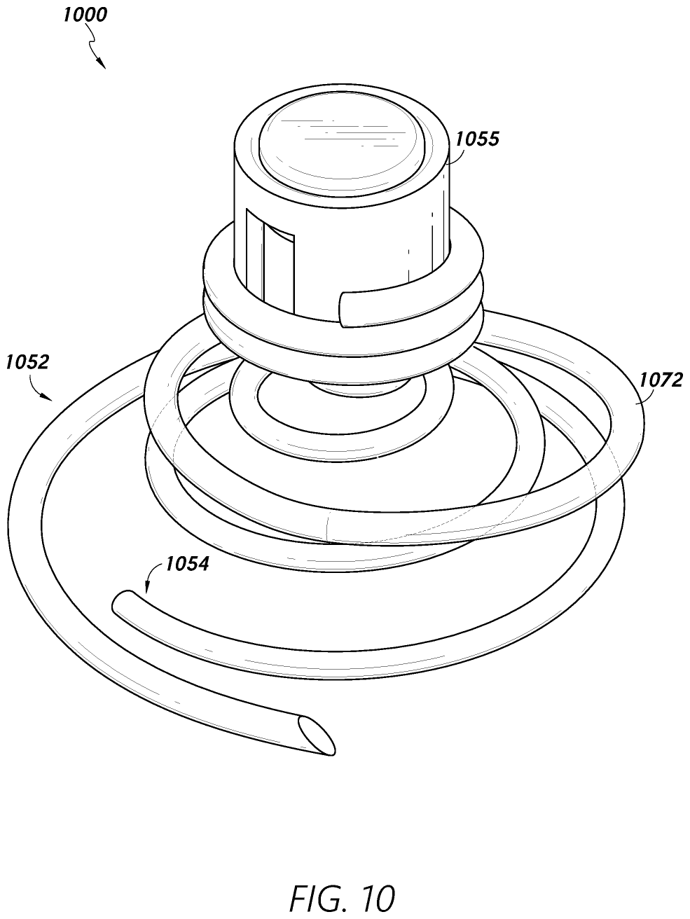

FIG. 11 shows a sensor implant device 1100 including primary 1152 and secondary 1154 anchors, wherein both the primary 1152 and secondary 1154 anchors have a cylindrical shape with respect to tissue-engagement portions thereof. That is, as with the embodiment shown in FIGS. 8A-9B, the secondary anchor 1154 can have a cylindrical/constant-diameter tissue-engagement portion moving from proximal to distal portions thereof. Furthermore, as an alternative to the illustrated embodiment shown in FIGS. 8A-9B, the primary anchor 1152 may likewise have a substantially constant diameter moving from proximal to distal portions thereof. The cylinder form/shape of the secondary anchor may be dimensioned and configured to fit within the cylinder form/shape of the primary anchor 1152 to allow for simultaneous presence and/or embedding of the respective anchors.

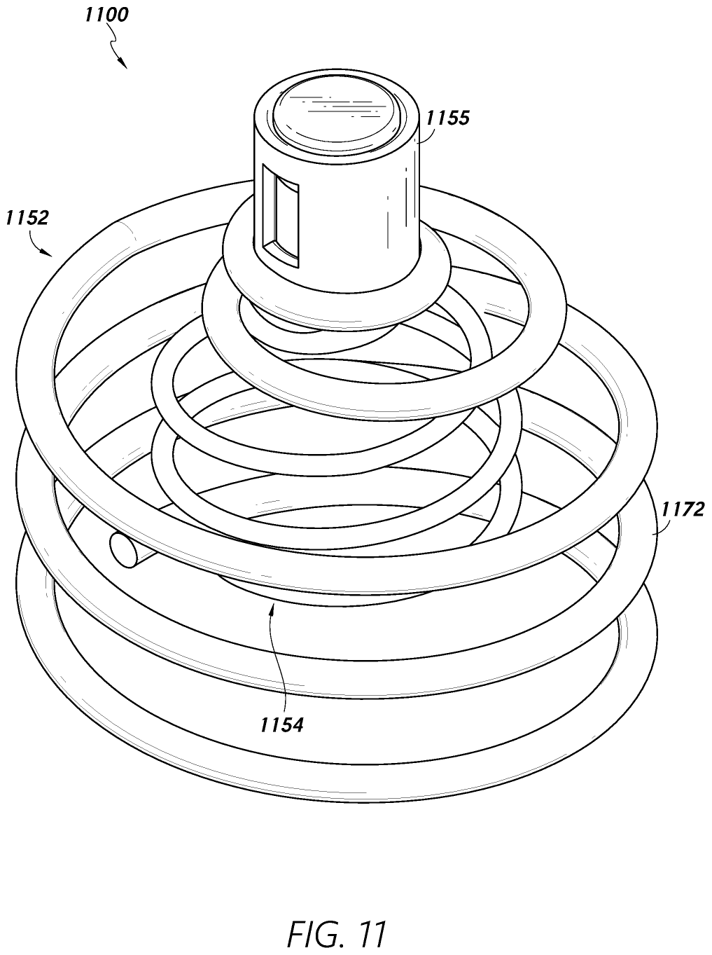

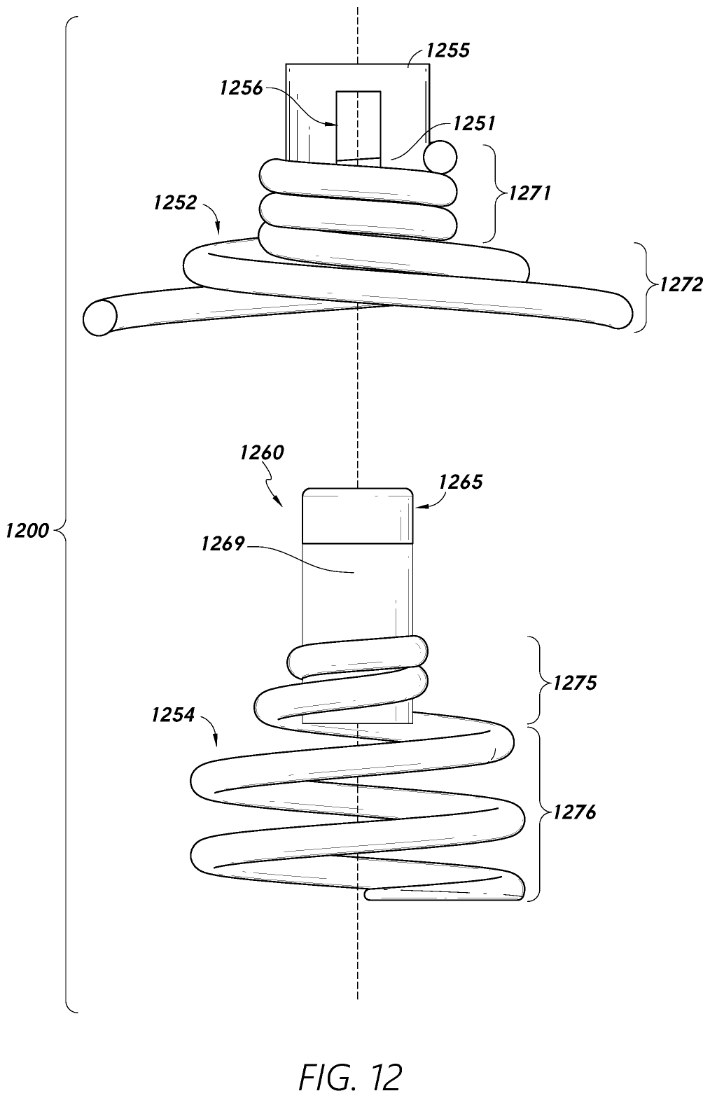

FIG. 12 shows an exploded view of an implant device 1200 including primary 1252 and secondary 1254 tissue anchors in accordance with embodiments of the present disclosure. In FIG. 12, the secondary anchor 1254 is shown as including an attachment portion 1275 that is configured to wrap around and/or otherwise be secured to the sensor housing 1269 of a sensor device 1260 of the implant device 1200. The sensor device 1260 may include a sensor element 1265 and a sensor housing 1269. Conversely, the primary anchor 1252 includes an attachment portion 1271 configured to be wrapped around and/or otherwise secured to an anchor base/housing 1255 of the sensor implant device 1200. That is, in some embodiments, the primary 1252 and secondary 1254 anchors can be attached to separate components of the device 1200, namely the anchor base/housing 1255 and the sensor housing 1269, respectively. In some embodiments, the primary anchor 1252 is attached to the sensor housing 1269 and/or the secondary anchor 1254 is attached to the anchor base/housing 1255.

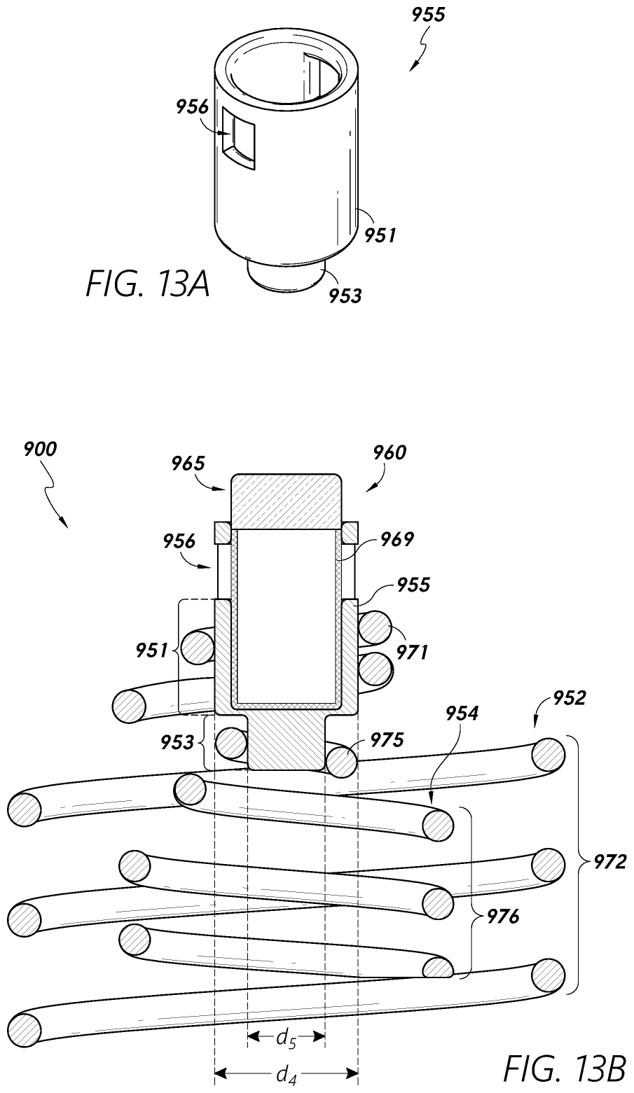

FIGS. 13A and 13B show an embodiment of a sensor implant device 900 including an anchor base/housing 955 configured to have both primary 952 and secondary 954 anchors attached thereto. The anchor baser/housing 955 may comprise a cylindrical form having a lumen, pocket, channel, or other internal feature configured to hold and/or have disposed at least partially therein a sensor housing 969 associated with a sensor device 960 of the sensor implant device 900.

The anchor base/rousing 955 further includes one or more exterior surfaces and/or features configured to have attached thereto respective attachment portions of primary 952 and secondary 954 anchors, wherein the primary and secondary anchors have opposing chirality, as described in detail herein. For example, the anchor base/housing 955 includes a primary anchor attachment portion 951, which is associated with a medial portion of the base/housing 955 and comprises a circumferential surface around which the attachment portion 971 of the primary anchor 952 can be wrapped or otherwise disposed and/or secured.

The anchor base/housing 955 further includes a secondary anchor attachment portion 953, which may be distal relative to the primary anchor attachment portion 951. The secondary anchor attachment portion 953 may likewise comprise a circumferential surface around which the attachment portion 975 of the secondary anchor 954 can be wrapped, disposed, or otherwise attached or secured.

In some embodiments, the primary anchor attachment portion 951 has a diameter d₄ that is greater than a diameter d₅ of the secondary anchor attachment portion 953. Such step-down diameter associated with the anchor base/housing 955 can allow for respective attachments of the primary anchor 952 and secondary anchor 954 while avoiding interference between the respective attachment portions thereof when both of the anchors are attached to the anchor base/housing 955. The cavity or lumen in which the sensor device 960 is disposed within the anchor base/housing 955 may or may not continue through the distal secondary anchor attachment portion 953. For example, the cavity/lumen may be distally closed, as shown in the cross-sectional view of FIG. 13. The distal ends of one or more of the attachment portions 951, 953 of the anchor base/housing 955 may have a flange or lip feature configured to retain the coils/wired in place. Such flange/lip features can act as stoppers configured to impede or prevent distal movement of the attachment portions of the coils/anchors such that they do not slide off distally from the anchor base/housing 955. In some embodiments, the anchor base/housing 955 is machined as a single piece. Alternatively, the anchor base/housing 955 can comprise a plurality of sleeves and/or other structures having different diameters that are welded or otherwise secured together.

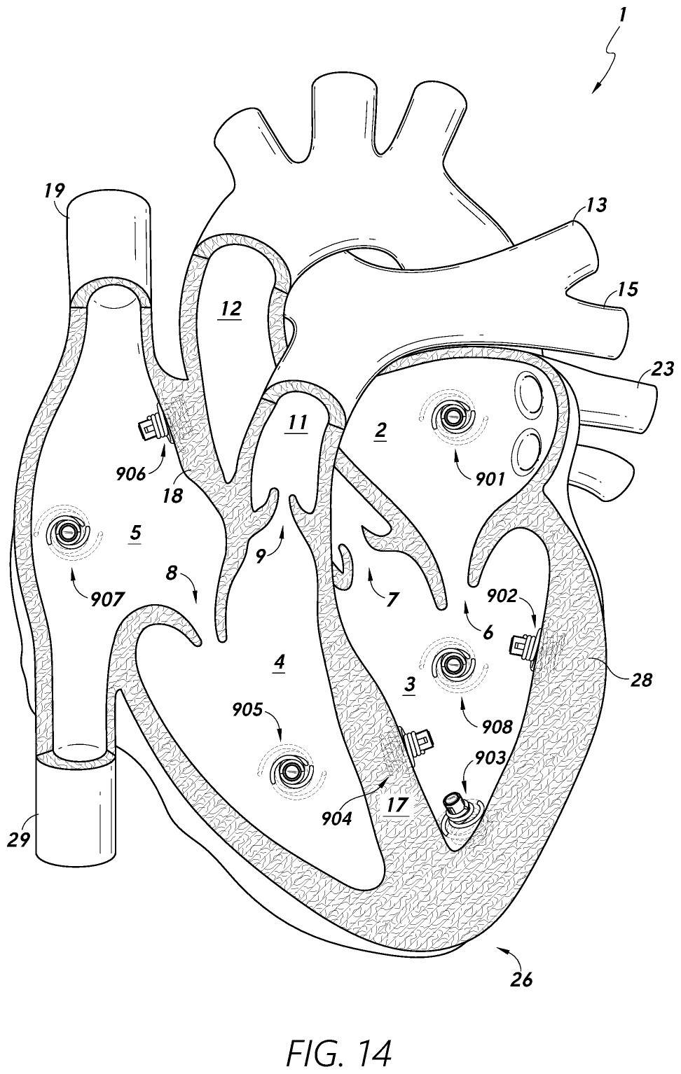

FIG. 14 shows a cutaway view of a heart 1 illustrating various example implantation positions for sensor implant devices in accordance with aspects of the present disclosure. It should be understood that sensor implant devices as disclosed herein may be implanted in any anatomy or material. However, FIG. 14 shows examples of anatomy that represent areas where implantation of sensor implant devices in accordance with aspects of the present disclosure can be advantageous.

The various implantation sites shown in FIG. 14 include implantation in the interatrial septum 18, as shown as example implant 906. Although shown in FIG. 14 as implanted in the interatrial septum 18 with the sensor transducer of the device 906 exposed in the right atrium 5, it should be understood that such implant location may be utilized, wherein the sensor transducer of the implant device is exposed on the left atrial side of the septum 18. Sensor implant devices in accordance with aspects of the present disclosure may further be implanted at other areas within the right atrium 5, such as in a wall of the right atrium as shown as example implant 907. It may be advantageous to implant the sensor implant device within the left atrium 2, such as in a tissue wall as shown at implant site 901.

The left ventricle 3 may further provide a chamber for implantation of sensor implant devices in accordance with aspects of the present disclosure. For example, a sensor implant device may be implanted in an outer ventricular wall, such as at the implantation site 902, or at or near the apex 26 of the heart 1, such as at the implantation site 903 shown. Another ventricular implantation site may be in the ventricular septum 17, as shown at example implant site 904. For example, a sensor implant device may be implanted in the septum 17 with the sensor transducer thereof exposed in the left ventricle 3 or the right ventricle 4. Example implant site 905 represents implantation within the right ventricle 4 in an area other than the ventricular septum 17, such as in an outer wall of the ventricle 4.

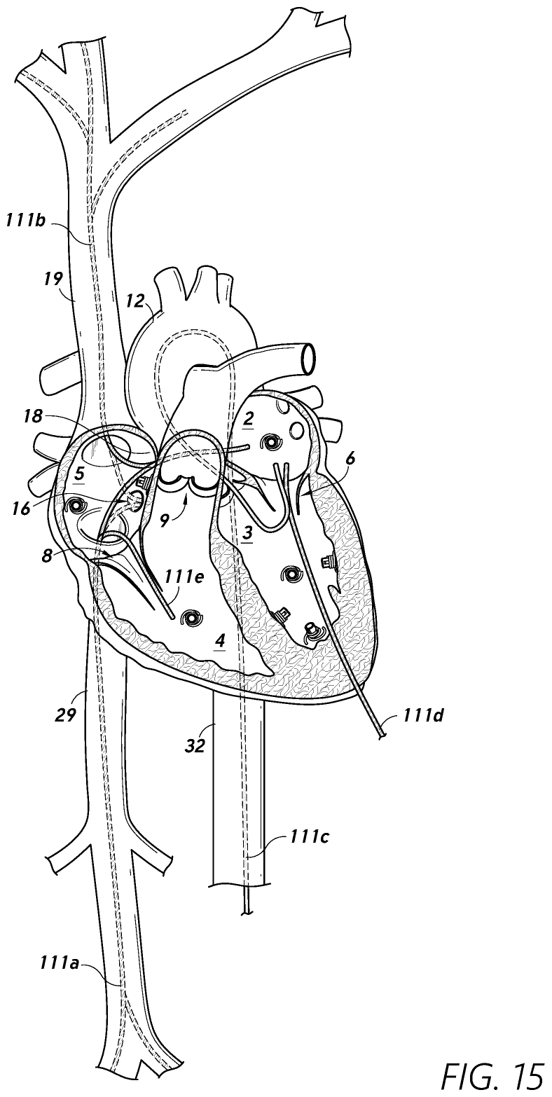

FIG. 15 shows various catheters 111 that may be used to implant sensor devices in accordance with aspects of the present disclosure. The catheters 111 can advantageously be steerable and relatively small in cross-sectional profile to allow for traversal of the various blood vessels and chambers through which they may be advanced en route to, for example, the right atrium 5, left, atrium 2 or other anatomy or chamber. Catheter access to the right atrium 5, coronary sinus 16, or left atrium 2 in accordance with certain transcatheter solutions may be made via the inferior vena cava 29 (as shown by the catheter 111a) or the superior vena cava 15 (as shown by the catheter 111b). Further access to the left atrium 2 may involve crossing the atrial septum 18 (e.g, in the area at or near the fossa ovalis).

Although access to the left atrium is illustrated and described in connection with certain examples as being via the right atrium and/or vena cavae, such as through a transfemoral or other transcatheter procedure, other access paths/methods may be implemented in accordance with examples of the present disclosure. For example, in cases in which septal crossing through the interatrial septal wall is not possible, other access routes may be taken to the left atrium 2. In patients suffering from a weakened and/or damaged atrial septum, further engagement with the septal wall can be undesirable and result in further damage to the patient. Furthermore, in some patients, the septal wall may be occupied with one or more implant devices or other treatments, wherein it is not tenable to traverse the septal wall in view of such treatment(s). As alternatives to transseptal access, transaortic access may be implemented, wherein a delivery catheter 111c is passed through the descending aorta 32, aortic arch 12, ascending aorta, and aortic valve 7, and into the left atrium 2 through the mitral valve 6. Alternatively, transapical access may be implemented to access the target anatomy, as shown by delivery catheter 111d.

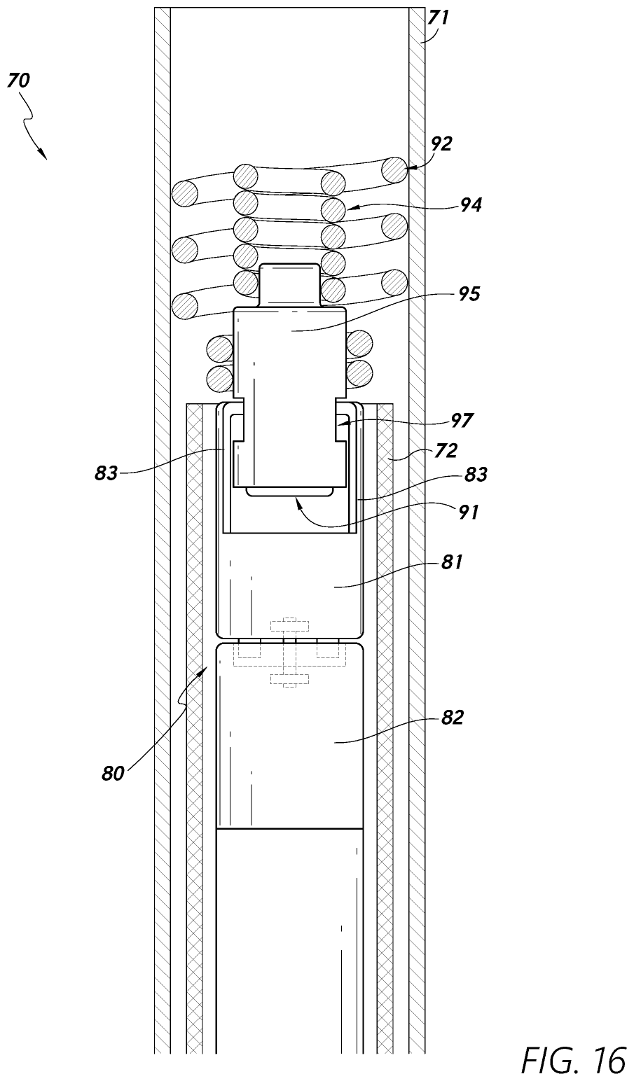

FIG. 16 illustrates a delivery system 70 for delivering and implanting a sensor implant device 90 in accordance with aspects of the present disclosure. The delivery system 70 includes a torquing shaft 80 configured to hold a sensor implant device 90 including a primary anchor 92 and a secondary anchor 94 coupled to an anchor base/housing 95, which holds a sensor device 96 as described in detail herein. The torquing shaft 80 may be configured to hold the sensor implant device 90 by engaging with one or more torque-engagement features 97 associated with the anchor base/housing 95. For example, the anchor base/housing 95 may include one or more apertures, windows, recesses, divots, edges, tabs, or other features configured to engage with one or more arms 83 of the torquing shaft 80 in a manner as to secure the base/housing 95 to the arms 83 and shaft 80 when the arms 83 is/are in a locked configuration or position, as shown in FIG. 16. The locking arms 83 may include distal inwardly-projecting ears/projections configured to nest within and/or otherwise engaged with the feature(s) 97.

The locking arms 83 may be configured to radially-inwardly engage the engagement feature(s) 97 of the base/housing 95. For example, the arms 83 may be brought over the outside of the housing/base 95, and radialy-inwardly actuated or permitted to engage with the engagement feature(s) 97. In some embodiments, an inner sheath 72 may be configured to hold the arms 83 in a radially inwardly-positioned configuration in a manner as to secure the arms 83 in the locked position shown in FIG. 16.

The torquing shaft 80 may be configured to be placed within and/or slide relative to the inner sheath 72. During delivery, the inner sheath 72 may be positioned over the locking arms 83 in order to secure the arms 83 to the base/housing 95 of the sensor implant device 90. Although shown with the inner sheath 72 positioned such that the distal end/opening thereof is near the distal end of the torquing shaft/arms 80/83, it should be understood that in some implementations, the inner sheath 72 may be configured such that one or both of the primary 92 and secondary 94 anchors are retained therein during transport. When the inner sheath 72 is disposed over the locking arms 83, the sheath 72 may hold the locking arms 83 in a mating engagement with the features 97 of the base/housing 95, thereby allowing for translation of torque from the locking arms 83 to the housing/base 95. By pulling back the inner sheath 72 relative to the torquing shaft 80, the locking arms 83 can be distally cleared of the sheath 72, thereby allowing the locking arms 83 to be released from the mating feature set 97 of the base/housing 95.

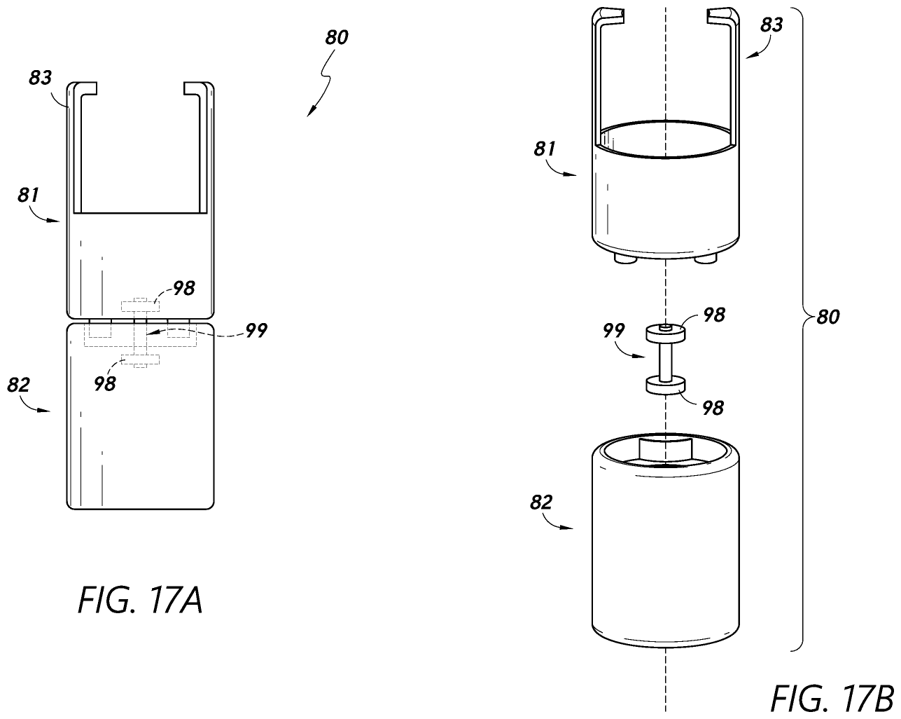

Unlocking of the locking arms 83 from engagement with the engagement features 97 of the base/housing 95 can be achieved by proximally pulling the inner sheath 72 and/or distally pushing the torquing shaft 80 such that the locking arms 83 are distally exposed past the distal end of the inner sheath 72, thereby allowing the locking arms 83 to radially deflect away from the torque-engagement features 97, thereby freeing the sensor implant device 90 and/or base/housing 95 thereof from engagement with the torquing shaft 80. In some embodiments, the torquing shaft 80 includes a distal torquing portion 81 and a torque-limiter portion 82, which are described in greater detail below in connection with FIGS. 17A-20D.

The delivery system 70 may further comprise an outer sheath 71, which may provide access to the implantation site, wherein the inner sheath 72 and/or shaft 80 may be inserted and/or retracted within the outer sheath 71. For example, at or near the implantation site, the inner sheath and torquing shaft 80 may be deployed from the distal end of the outer sheath 71 and retracted back into the outer sheath 71 after implantation of the sensor implant device 90.

Although shown in a coiled configuration, in some implementations, the primary 92 and/or secondary 94 tissue anchors may be disposed in a relatively elongated/stretch configuration in the delivery system 70 during transport. The primary 92 and secondary 94 tissue anchor coils may be compressed to fit within the outer sheath 71. For example, the coils may be wound more tightly in the delivery configuration shown in FIG. 16 compared to the deployed, on constrained configuration thereof.

FIGS. 17A and 17B show side and exploded views of a torquing shaft 80 in accordance with one or more embodiments. The torquing shaft 80 may be similar in one or more respects to the torquing shaft shown in FIG. 16 and described above. In some embodiments, the torquing shaft 80 is configured to prevent torquing/rotation of the device beyond what is desirable with respect to the target tissue and/or relevant sensor implant device. In order to achieve such functionality, the torquing shaft 80 can include a distal torquing portion 81 and a torque-limiter portion 82, as shown. The distal torquing portion 81 can be coupled to the proximal torque-limiter portion 82 in a rotatable manner. The distal torquing portion 81 can be associated with locking arms 83, which are described in greater detail above. The torquing portion 81 of the torquing shaft 80 can comprise a tube that is broken at the end to accommodate radial deflection of the locking arms 83. For example, one or more (e.g., two) strips/arms may be cut from the tube to form the locking arms 83.

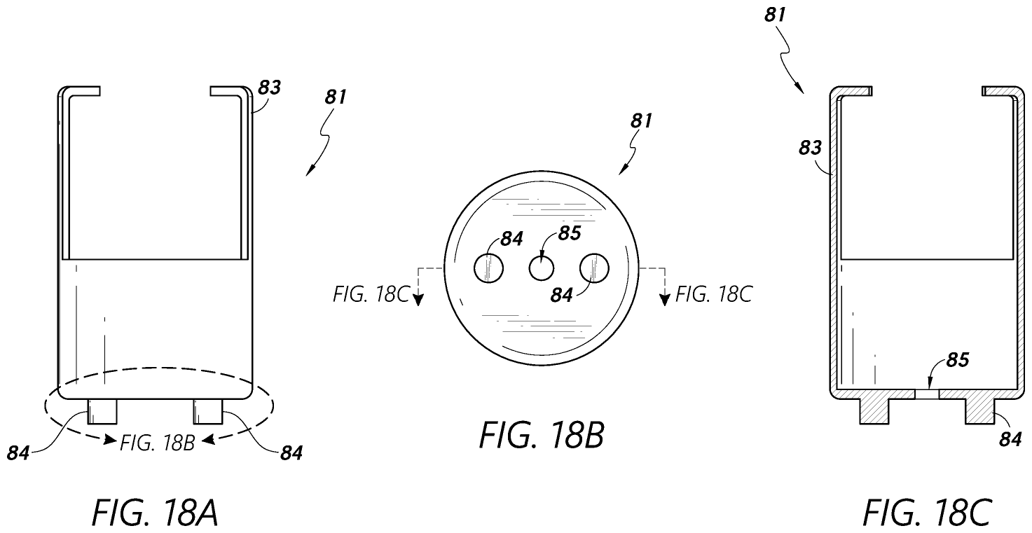

A pin 99 or other rotational coupling means may be coupled to both the distal torquing portion 81 and the torque-limiter portion 82, wherein one or both of the portions 81, 82 can be configured to rotate axially about the pin 99. For example, the pin 99 may be inserted through an aperture associated with one or both of the shaft portions 81, 82, wherein the portions 81, 82 can rotate relative to one another about the axis of the pin 99 and/or aperture(s). The pin 99 may be axially secured to the portions 81, 82, with one or more flanges, nuts, studs, buttons, washers, sockets, or other axial-retention stopper features 98, which may be coupled to the pin 99 in some manner within the respective portions 81, 82 of the torquing shaft 80. For example, such axial retention stopper features 98 may prevent the torquing shaft portions 81, 82 from sliding off of the pin 99, and may thereby hold the two portions 81, 82 together in a mechanical coupling, wherein the portions 81. 82 may still be permitted to rotate relative to one another about the pin. Details relating to the rotational coupling of the torquing portion 81 and the torque-limiting portion 82 of the torquing shaft 80 are described below in connection with FIGS. 18A-18C, 19A-19C, and 20A-20D.

FIGS. 18A-18C show side, cross-sectional, and axial views, respectively, of a torquing portion 81 of a torquing shaft according to aspects of the present disclosure. The torquing portion 81 includes locking arms 83 at or near a distal end thereof, whereas a proximal end of the torquing portion 81 includes certain features for interfacing with, and rotating relative to, the torque-limiter portion 82 (see FIGS. 17A and 17B). For example, at or near a proximal end of the torquing portion 81, such as on or associated with a proximal face or end, and axial aperture 85 or other feature may be present, wherein such feature(s) may facilitate rotation of the torquing portion 81 relative to the torque-limiter portion 82. The proximal end of the torquing portion 81 may further include one or more raised interference pegs 84, which may generally project proximally and may serve to provide mechanical resistance between the torquing portion 81 and the coupled torque-limiter portion 82 when the portions are rotated relative to one another, to thereby facilitate the transfer of torque between such components. The interference peg(s) 84 may have an axis that is parallel to an axis of the torquing portion 81.

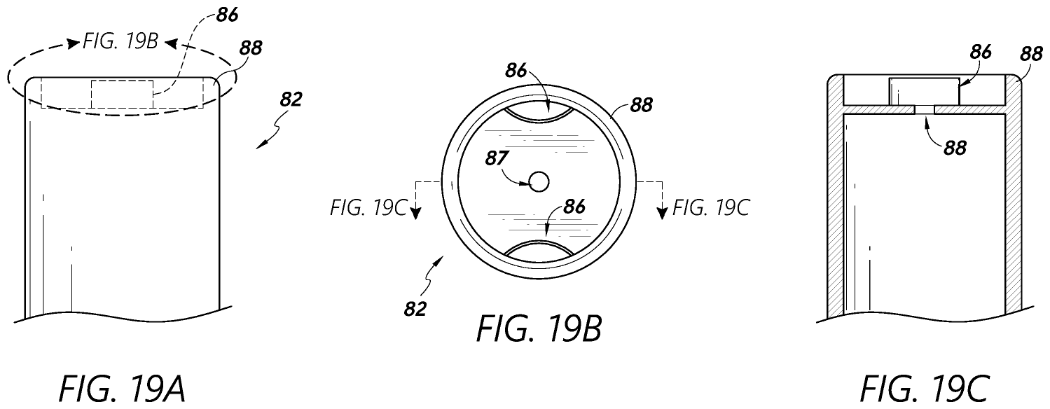

FIGS. 19A-19C shows side, cross-sectional, and axial views, respectively, of the torque-limiter portion 82 of the torquing shaft 80 according to one or more embodiments of the present disclosure. The torque-limiter portion 82 is configured to be coupled to the torquing portion 81. For example, the distal end of the torque-limiter portion 82 can have associated therewith an axial aperture 87, which may be configured to have disposed at least partially therein a pin about which one or more of the portions of the torquing shaft can rotate. Although both the torquing portion 81 and the torque-limiter portion 82 are illustrated and described as having an aperture for a pin, in some embodiments, only one portion includes an aperture, whereas the pin may be fixed to and/or integrated with the other portion.

The torque-limiter portion 82 may further include one or more deflection plates, panels, or other forms 86, which are configured to provide mechanical resistance against the interference pegs 84 of the torquing portion 81 when the torquing portion 81 is rotated relative to the torque-limiter portion in a manner as to bring the interference pegs 84 into physical contact with the deflection plates/forms 86. In some embodiments, the deflection forms 86 comprise metal or plastic strips, plates, panels, or other forms having a shape that is configured to resist inward deflection or deformation thereof with respect to a curvature of the deflection forms 86 to resist deflection from contact with the interference peg(s) 84 of the torquing portion 81 in order to translate rotational force from the deflection forms 86 to the interference pegs 84 when brought into contact therewith up to an amount commensurate with the resistance of the deflection forms 86.

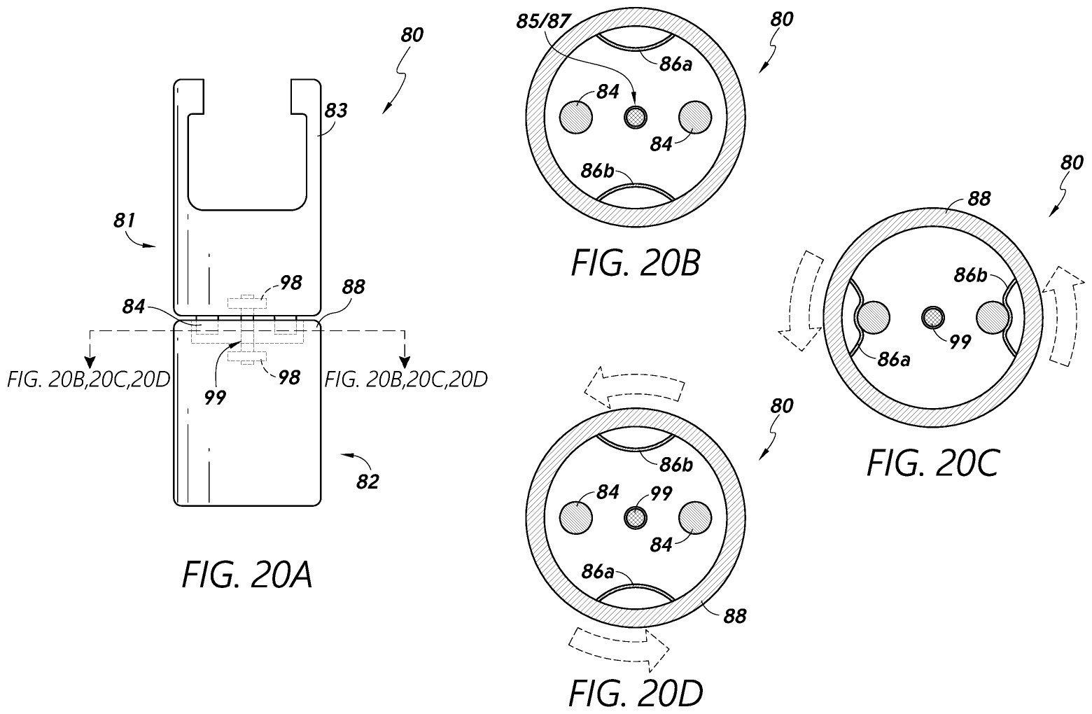

FIGS. 20A-20D illustrate the torque-limiting interface between the torquing portion 81 and the torque-limiter portion 82 according to one or more embodiments of the present disclosure. Specifically, FIG. 20A shows a side view of a coupling between a torquing portion and a torque-limiting portion of a torquing shaft in accordance with one or more embodiments. FIGS. 20B-20D show axial views of a coupling between a torquing portion and a torque-limiting portion of a torquing shaft in various states in accordance with one or more embodiments.

FIG. 20B shows the interface between the torquing portion 81 and the torque-limiter portion 82 in a configuration in which the interference pegs 84 are not in contact with the deflection forms 86. With the pegs 84 not in contact with the deflection forms 86, the deflection forms 86 will not translate rotational force to the torquing portion via the interference pegs 84 when the torque-limiting portion 82 is rotated.

FIG. 20C shows the interface between the torquing portion 81 and the torque-limiter portion 82 after approximately 90° of rotation relative to the configuration shown in FIG. 20B, wherein such rotation has brought the interference pegs 84 into contact with the deflection forms 86. In such a configuration, further application of rotational force to the torque-limiter portion 82 in an amount that does not exceed the resistance of the deflection forms 86 with respect to the interference pegs 84 can result in a commensurate rotational force being applied to the interference pegs 84, and therefore to the torquing portion 81, thereby causing rotation of the torquing portion 81 to rotate the sensor implant device. Such rotation may serve to embed the primary anchor held by the torquing shaft into the target tissue.

FIG. 20D shows the interface between the torquing portion 81 and the torque-limiter portion 82 after further rotational force has been applied to the torque-limiter portion 82 in an amount in excess of the resistance threshold of the deflection forms 86 with respect to contact with the interference pegs 84. Such rotational force can cause the deflection forms to rotate past the interference pegs 84, such that rotation of the torque-limiter portion 82 is not translated to the torquing portion 81. Therefore, the particular resistance of the deflection forms 86 may be selected and/or configured to limit the amount of torque that can be translated from the torque-limiter portion 82 to the torquing portion 81, to limit over-torquing of the sensor implant device and/or associated anchor(s) to prevent damage to anatomy and/or device(s). Although the interference pegs 84 are described as being associated with the torquing portion 81 and the deflection forms 86 are described as being associated with the torque-limiter portion, it should be understood that in some implementations, the interference pegs 84 are associated with the torque-limiter portion 82 and the deflection forms 86 are associated with the torquing portion 81.

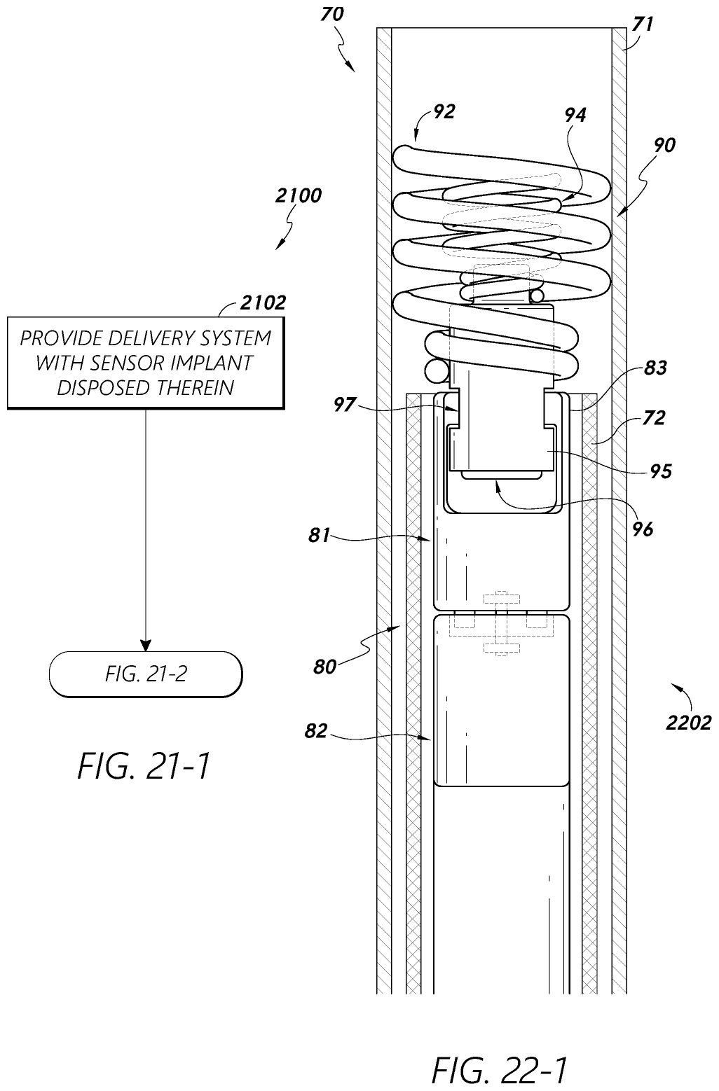

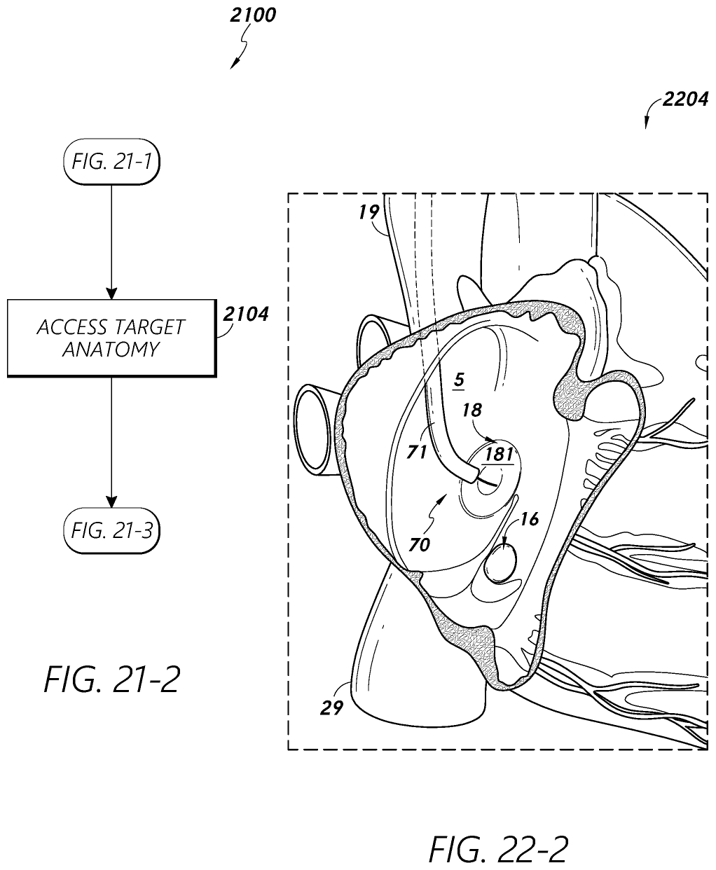

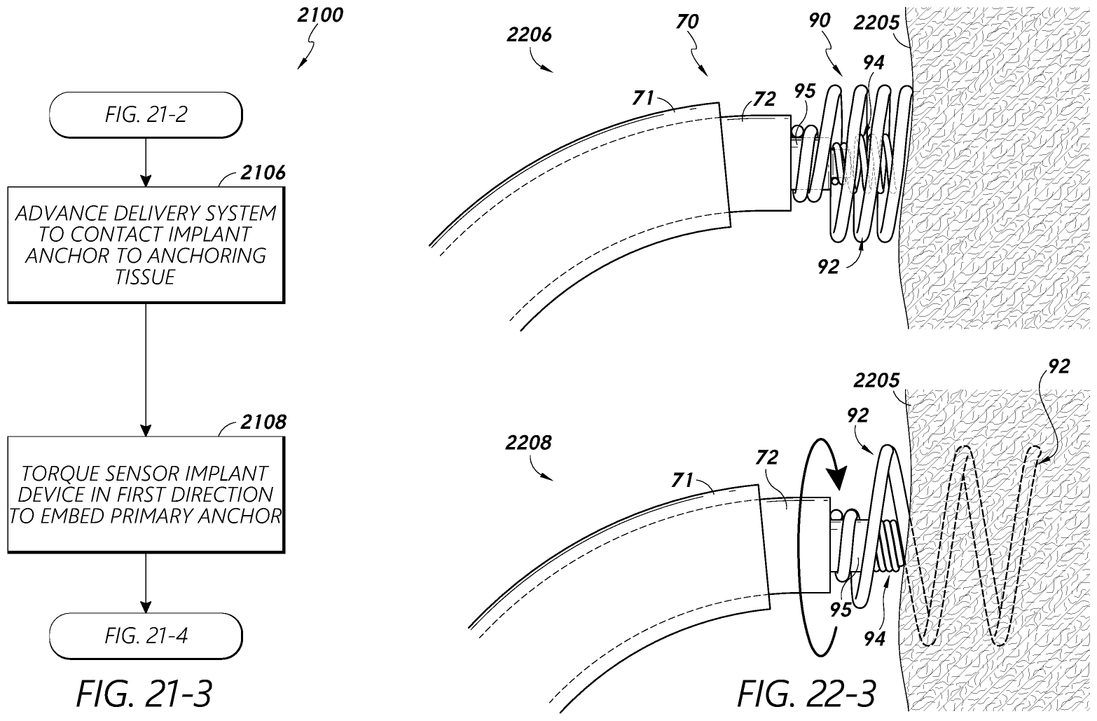

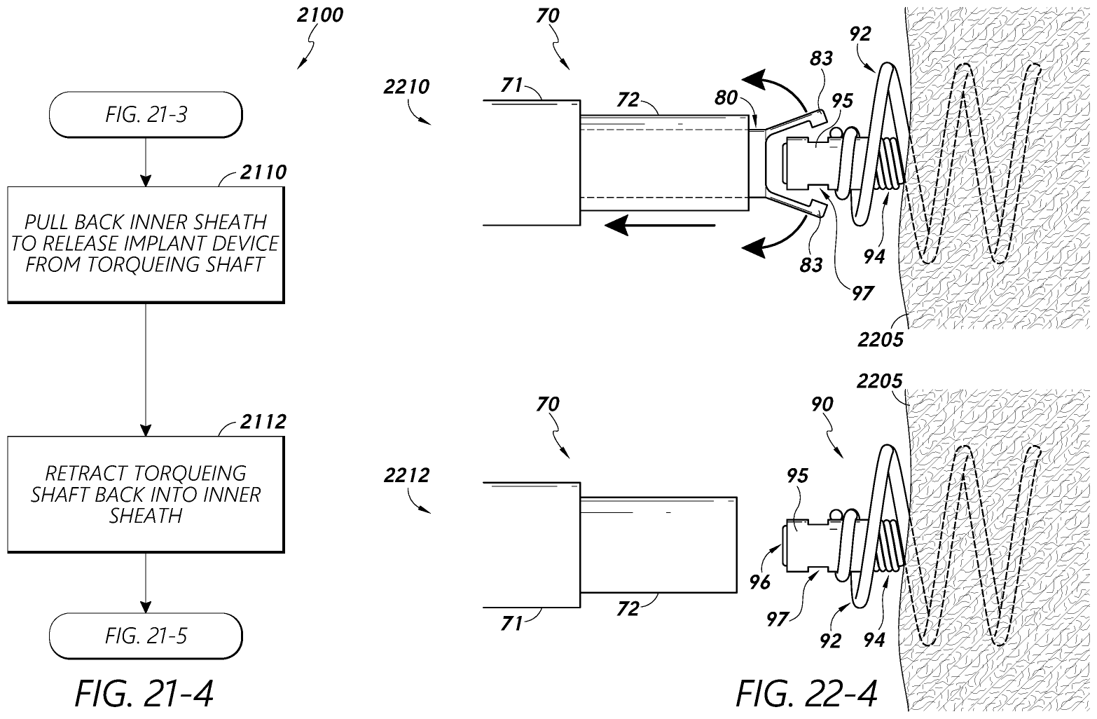

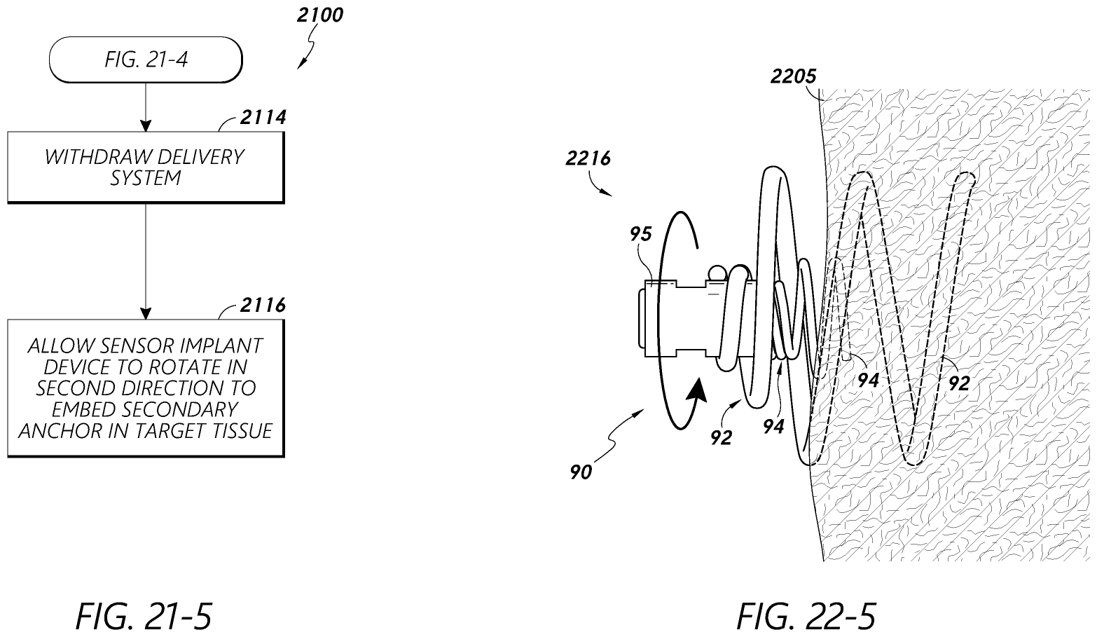

FIGS. 21-1, 21-2, 21-3, 21-4, and 21-5 provide a flow diagram illustrating a process 2100 for implanting a sensor implant device in accordance with one or more embodiments. FIGS. 22-1, 22-2, 22-3, 22-4, and 22-5 provide images of cardiac anatomy and certain devices/systems corresponding to operations of the process 2100 of FIGS. 21-1, 21-2, 21-3, 21-4, and 21-5 in accordance with one or more embodiments.

At block 2102, the process 2100 involves providing a delivery system 70 with a sensor implant device 90 disposed therein in a delivery configuration. Image 2202 of FIG. 22-1 shows a partial cross-sectional view of the delivery system 70 and sensor implant device 90 in accordance with one or more embodiments of the present disclosure. The image 2202 shows the sensor implant device 90 disposed within an outer sheath 71 of the delivery system 70. Although a particular embodiment of a delivery system is shown in FIG. 22-1, it should be understood that sensor implant devices in accordance with aspects of the present disclosure may be delivered and/or implanted using any suitable or desirable delivery system and/or delivery system components. The delivery system 70 may be similar to the delivery system shown in FIG. 16 and described above in one or more respects.

The illustrated delivery system 70 includes an inner sheath/catheter 72, which may be disposed at least partially within the outer sheath 71 during one or more periods of the process 2100. In some embodiments, the delivery system 70 may be configured such that a guidewire may be disposed at least partially therein. For example, the guidewire may run in the area of an axis of the sheath 71 and/or inner catheter 72, such as within the inner catheter 72. The delivery system 70 may be configured to be advanced over the guidewire to guide the delivery system 55 to a target implantation site.

The outer sheath 71 may be used to transport the sensor implant device 90 to the target implantation site. That is, the sensor implant device 90 may be advanced to the target implantation site at least partially within a lumen of the outer sheath 71, such that the sensor implant device 90 is held and/or secured at least partially within a distal portion of the outer sheath 71.

At block 2104, the process 2100 involves accessing a target site/anatomy with the delivery system 70. For example, such access may be made through a transcatheter access path, such as described herein. In some embodiments, the target anatomy is a chamber of the heart of the patient, for example. In some implementations, access to the target implantation site may be facilitated using a guidewire. For example, a guidewire may be disposed within the delivery system 70, such as within the torquing shaft 80 and through the sensor implant device. For example, the sensor transducer 91 of the sensor device 96 can have an axial hole that is not covered by the sensor transducer, forming a torus-shaped sensor membrane. A guidewire may also be run through the inside of the coils of the primary 92 and/or secondary 94 tissue anchors.

At block 2106, process 2100 involves advancing the delivery system 70 and/or one or more components thereof to contact a primary anchor 92 of an implant device 90 associated with the delivery system 70 to target tissue 2205. For example, where the target tissue is in the left atrium, transseptal access may be implemented to advance the delivery system to the target tissue. Access to the septum and left atrium via the right atrium may be achieved using any suitable or desirable procedure. In sonic embodiments, access may be achieved through the subclavian or jugular vein into the superior vena cava (not shown) and from there into the right atrium. Alternatively, the access path may start in the femoral vein and through the inferior vena cava (not shown) into the heart. Other access routes may also be used, each of which may typically utilize a percutaneous incision through which the guidewire and catheter are inserted into the vasculature, normally through a sealed introducer, and from there the system may be designed or configured to allow the physician to control the distal ends of the devices from outside the body.

In some implementations, a guidewire is introduced through the subclavian or jugular vein, through the superior vena cava, and into the right atrium. In some implementations, the guidewire can be disposed in a spiral configuration within the left atrium, which may help to secure the guidewire in place. Once the guidewire provides a path, an introducer sheath may be routed along the guidewire and into the patient's vasculature, such as with the use of a dilator. The delivery catheter 70 may be advanced through the superior vena cava to the right atrium, wherein the introducer sheath may provide a hemostatic valve to prevent blood loss. In some embodiments, a deployment catheter may function to form and prepare an opening in the septum, and a separate placement delivery system is used for delivery of the sensor implant device 90. In other embodiments, the delivery system 70 may be used as the both the puncture preparation and implant delivery catheter with full functionality. In the present application, the term “delivery system” is used to represent a catheter or introducer with one or both of these functions.

At block 2108, the process 2100 involves torquing the sensor implant device 90 and/or associated primary anchor 92 in a first direction corresponding to a chirality of the primary anchor 92. Such torquing of the sensor implant device may be implemented using a torquing shaft 80 mechanically coupled to the sensor implant device. For example, the torquing shaft may include one or more locking arms 83 or other engagement features configured to be engaged with corresponding features of a housing or other structure associated with the sensor implant device 90. The operations associated with block 2108 may involve torquing the sensor implant device 90 until a desired depth of penetration of the primary anchor 92 is achieved. For example, monitoring of the depth of embedding may be performed using any type of imaging technology/modality.

The secondary anchor 94 of the sensor implant device may have a chirality that is opposite the chirality of the primary tissue anchor 92. Therefore, rotation in the first direction corresponding to the chirality of the primary anchor 92 may generally not result in the secondary anchor 94 embedding in the tissue wall 2205. Rather, the puncture tip of the secondary anchor 94 may be dragged along the surface of the target tissue 2205 without the tip embedding into the target tissue to a substantial degree.

In some implementations, the primary 92 and secondary 94 tissue anchors may be compressed against the target tissue 2205 and the sensor implant device may be rotated in a direction in accordance with the chirality of the primary anchor 92 of the sensor implant device 90 to cause the primary anchor 92 to bite into the target tissue, whereas the secondary anchor 94 may be compressed between the surface of the target tissue 2205 and the sensor housing 95 where the secondary sensor 94 is proximally attached to the sensor implant device 90.

At block 2110, the process 2100 involves pulling hack an inner sheath 72 of the delivery system 70 to expose the locking arms 83 of the torquing shaft 80, thereby allowing the locking arms 83 to disengage from the corresponding engagement features of the sensor implant device 90 to thereby release the sensor implant device 90 from the torquing shaft 80 and/or delivery system 70. Once released, the locking arms 83 may deflect radially outwardly to disengage from the anchor base/housing 95. At block 2112, the process 2100 involves retracting the torquing shaft 80 back into the inner sheath 72, to thereby bring the locking arms back into a compressed configuration.

At block 2114, the process 2100 involves withdrawing the delivery system 70, thereby leaving the sensor implant device 90 implanted at the target implantation site. At block 2116, the process 2100 involves allowing the sensor implant device 90 to rotate in a second direction opposite the first direction, wherein the second direction corresponds to a chirality of the secondary anchor 94, thereby at least partially embedding the secondary anchor 94 in the target tissue 2205. Such rotation of the sensor implant device 90 in the second direction may be caused at least in part by turbulence and/or movement at the implantation site associated with the normal cardiac rhythm of the heart. In some embodiments, rotation in the second direction may be caused at least in part by spring force of the secondary anchor 94 pushing away from the target tissue 2205, which may be a result of the secondary anchor 94 having become at least partially compressed due to the embedding of the primary anchor 92 and associated approximation of the sensor implant device 90 to the target tissue 2205 resulting therefrom. Such compression may result in increased potential energy in the secondary anchor coils causing force against the tissue surface, thereby pushing the sensor implant device 90 away from the tissue surface 2205 and causing some amount of unwinding and/or backing-out of the primary tissue anchor 92. The unwinding/backing-out of the primary anchor 92 can result in rotation of the sensor implant device 90 in a direction opposite the direction of chirality of the primary tissue anchor 92. Such rotation can cause the tip of the secondary anchor to embed in the target tissue and to wind into the target tissue by some amount. Therefore, the unwinding/backing-out of the primary anchor 92 can serve to further secure the sensor implant device in place by embedding the secondary anchor 94, which can impede/prevent further unwinding/backing-out of the primary anchor 92 and dislodgement of the sensor implant device 90.

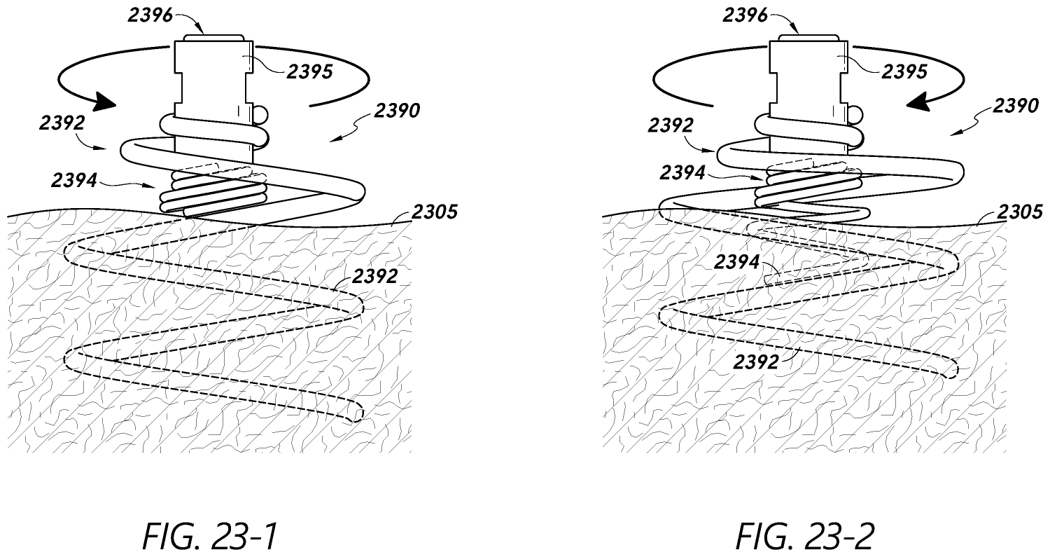

FIGS. 23-1 and 23-2 illustrate various implantation stages/states for a sensor implant device 2390 in accordance with one or more aspects of the present disclosure. In the implantation implementation shown in FIGS. 23-1 and 23-2, the primary anchor 2392 is a radially outer anchor of the primary and secondary anchor assembly. Therefore, according to the implantation implementation of FIGS. 23-1 and 23-2, the implant device 2390 may initially be embedded in the target tissue 2305 by rotating the implant device 2390 in accordance with the chirality of the outer anchor 2392, thereby causing the outer anchor 2392 to embed in the target tissue 2305, as shown in FIG. 23-1. The sensor implant device 2390 includes a sensor device 2396.

The inner anchor 2394 serves as the secondary anchor with respect to the anchor assembly of the sensor implant device 2390. When the sensor implant device 2390 is rotated in accordance with the chirality of the outer anchor 2392, such that the outer anchor 2392 embeds in the target tissue 2305, the inner/secondary anchor 2394, which may be secured to the housing 2395 and/or other structure of the sensor implant device 2390, may be compressed and/or held in a compressed state against the target tissue 2305, as shown in FIG. 23-1. Such compression may exert a force substantially normal to and/or away from the surface of the target tissue 2305. The force of the inner/secondary anchor 2394 may result in at least partial unwinding /backing-out of the outer primary anchor 2392 and commensurate embedding of the inner secondary anchor 2394, as shown in FIG. 23-2. Additionally or alternatively, the unwinding/backing-out of the outer/primary anchor 2392 and/or associated embedding of the inner; secondary anchor 2394 may result from motion and/or fluid dynamics associated with the target tissue 2305 and/or implantation site/environment.

FIG. 23-2 shows the secondary/inner anchor 2394 at least partially embedded in the target tissue 2305 after the outer anchor 2392 has been embedded in the target tissue 2305. The embedding of the inner/secondary anchor 2394 can restrict further unwinding of the outer primary anchor 2392, as described in detail in connection with various embodiments and implementations disclosed herein.

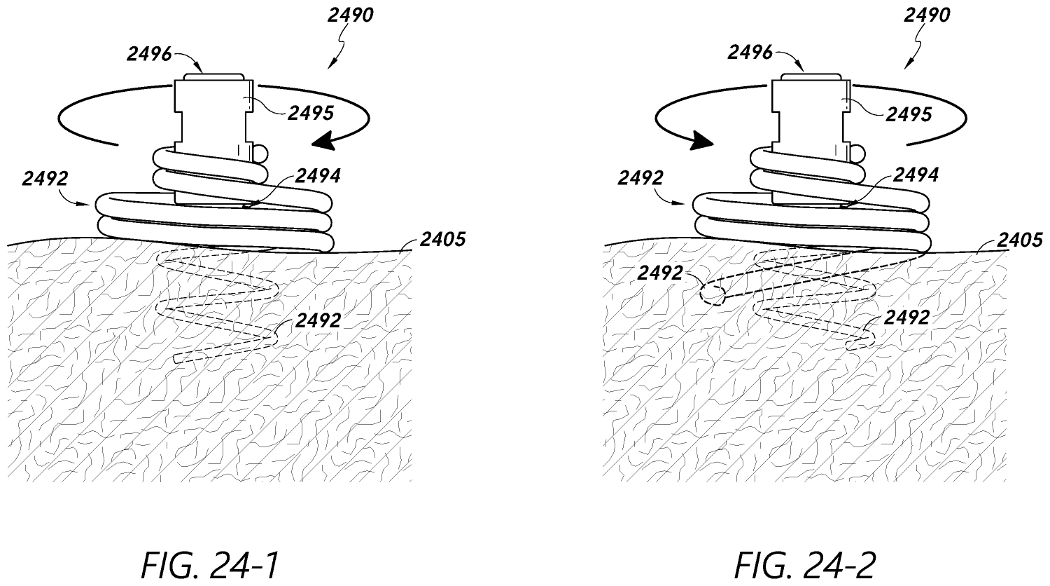

FIGS. 24-1 and 24-2 illustrate various implantation stages/states for a sensor implant device 2490 in accordance with one or more aspects of the present disclosure. In the implantation implementation shown in FIGS. 24-1 and 24-2, the primary anchor 2494 is a radially inner anchor of the primary and secondary anchor assembly. Therefore, according to the implantation implementation of FIGS. 24-1 and 24-2, the implant device 2490 may initially be embedded in the target tissue 2405 by rotating the implant device 2490 in accordance with the chirality of the inner anchor 2494, thereby causing the inner anchor 2494 to embed in the target tissue 2405, as shown in FIG. 24-1. The sensor implant device 2490 includes a sensor device 2496.

The outer anchor 2492 serves as the secondary anchor with respect to the anchor assembly of the sensor implant device 2490. When the sensor implant device 2490 is rotated in accordance with the chirality of the inner anchor 2494, such that the inner anchor 2494 embeds in the target tissue 2405, the outer/secondary anchor 2492, which may be secured to the housing 2495 and/or other structure of the sensor implant device 2490, may be compressed and/or held in a compressed state against the target tissue 2405, as shown in FIG. 24-1. Such compression may exert a force substantially normal to and/or away from the surface of the target tissue 2405. The force of the outer/secondary anchor 2492 may result in at least partial unwinding /backing-out of the inner primary anchor 2494 and commensurate embedding of the outer secondary anchor 2492, as shown in FIG. 24-2. Additionally or alternatively, the unwinding/backing-out of the inner/primary anchor 2494 and/or associated embedding of the outer/secondary anchor 2492 may result from motion and/or fluid dynamics associated with the target tissue 2405 and/or implantation site/environment.

FIG. 24-2 shows the secondary/inner anchor 2494 at least partially embedded in the target tissue 2405 after the outer anchor 2492 has been embedded in the target tissue 2405. The embedding of the outer/secondary anchor 2494 can restrict further unwinding of the outer primary anchor 2492, as described in detail in connection with various embodiments and implementations disclosed. herein.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A sensor implant device comprising:
a sensor device;
an anchor base structure secured to the sensor device;
a first helical tissue anchor secured to at least one of the sensor device or the anchor base structure, the first helical tissue anchor winding in a first direction; and
a second helical tissue anchor secured to at least one of the sensor device or the anchor base structure, the second helical tissue anchor winding in a second direction opposite the first direction.

2. The sensor implant device of claim 1, further comprising a third helical tissue anchor secured to at least one of the sensor device or the anchor base structure, the third helical tissue anchor winding in the first direction.

3. The sensor implant device of claim 2, wherein a tip of the first helical tissue anchor is positioned at a first circumferential position and a tip of the third helical tissue anchor is positioned at a second circumferential position with respect to an axis of the sensor device, the second circumferential position being circumferentially offset from the first circumferential position.

4. The sensor implant device of claim 3, wherein the tip of the first helical tissue anchor is positioned opposite the tip of the third helical tissue anchor with respect to a radius of the sensor implant device.

5. The sensor implant device of claim 1, wherein the anchor base structure comprises one or more torque engagement features.

6. The sensor implant device of claim 5, wherein the one or more torque engagement features comprises one or more radial apertures.

7. The sensor implant device of claim 1, wherein the first helical tissue anchor has a first diameter, the second helical tissue anchor has a second diameter; and the first diameter is greater than the second diameter.

8. The sensor implant device of claim 1, wherein a distal portion of the first helical tissue anchor has a greater pitch than a distal portion of the second helical tissue anchor.

9. The sensor implant device of claim 1, wherein the anchor base structure comprises:
a first anchor attachment portion configured to have an attachment portion of the first helical tissue anchor attached thereto; and
a second anchor attachment portion configured to have an attachment portion of the second helical tissue anchor attached thereto.

10. The sensor implant device of claim 9, wherein the first anchor attachment portion has a diameter that is greater than a diameter of the second anchor attachment portion.

11. The sensor implant device of claim 9, wherein the first anchor attachment portion is associated with a medial portion of the anchor base structure, and the second anchor attachment portion is associated with an end portion of the anchor base structure.

12. A sensor implant device comprising:
a sensor device comprising a sensor transducer and a wireless transmitter;
a first tissue anchor means secured to the sensor device, the first tissue anchor means having a first chirality; and
a second tissue anchor means secured to the sensor device, the second tissue anchor means having a second chirality that is opposite the first chirality.

13. The sensor implant device of claim 12, wherein at least one of the first tissue anchor means or the second tissue anchor means is secured to the sensor device via an anchor base structure.

14. The sensor implant device of claim 13, wherein the anchor base structure is secured to a body of the sensor device.

15. The sensor implant device of claim 13, wherein the anchor base structure comprises a torque-engagement means.

16. The sensor implant device of claim 15, wherein the torque-engagement means comprises at least one of a radial engagement aperture, recess, or edge.

17. The sensor implant device of claim 16, wherein the second tissue anchor means is disposed, at least in part, radially within the first tissue anchor means.

18. The sensor implant device of claim 16, wherein:
the first tissue anchor means and second tissue anchor means are corkscrew anchors;
the first tissue anchor means is conical; and
the second tissue anchor means is cylindrical.

19. A method of implanting a sensor implant device, the method comprising:
advancing a delivery system to a target tissue wall, the delivery system comprising:
a sensor implant device including a housing structure having one or more torque engagement features;
a first helical tissue anchor secured to the housing structure, the first helical tissue anchor winding in a first direction;
a second helical tissue anchor secured to the housing structure; the second helical tissue anchor winding in a second direction opposite the first direction; and
a torquing shaft including one or more locking arms configured to engage with the one or more torque engagement features of the housing structure;
rotating the torquing shaft in the first direction to at least partially embed the first helical tissue anchor in the target tissue wall; and
permitting the sensor implant device to rotate in the second direction to thereby at least partially withdraw the first helical tissue anchor from the target tissue wall and embed the second helical tissue anchor in the target tissue wall.

20. The method of claim 19, further comprising retracting a sheath from around a distal portion of the torquing shaft to cause the one or more locking arms to disengage from the one or more torque engagement features of the housing structure.
